(19) United States
(12) Patent Application Publication
(10) Pub. No.: US 2010/0130500 A1
(43) Pub. Date: May 27, 2010

(54) METHODS AND COMPOSITIONS FOR THE TREATMENT OF PULMONARY HYPERTENSION OF THE NEWBORN

(75) Inventor: Emil D. Kakkis, Novato, CA (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: BIOMARIN PHARMACEUTICAL INC., Novato, CA (US)

(21) Appl. No.: 11/720,815

(22) PCT Filed: Dec. 8, 2005
(86) PCT No.: PCT/US05/44587

§ 371 Date: May 8, 2008

Related U.S. Application Data
(60) Provisional application No. 60/634,147, filed on Dec. 8, 2004.

(51) Int. Cl. A61K 31/4985; A61P 9/12

(52) U.S. Cl. 514/249

(57) ABSTRACT

The present invention is directed to a novel methods and compositions for the therapeutic intervention in persistent pulmonary hypertension of the newborn (PPHN). More specifically, the specification describes methods and compositions for treating various types of PPHN using compositions comprising BH4. Combination therapies of BH4 and other therapeutic regimens are contemplated.

### BACKGROUND

1. Field of the Invention

The present invention is generally directed to the therapeutic intervention of respiratory disease of newborn. More particularly, the present invention is directed to methods and compositions for the treatment of persistent pulmonary hypertension of the newborn (PPHN).

2. Background of the Related Art

The transition from fetal to postnatal circulation occurs in four phases: (1) During the in-utero phase before birth, the fetal pulmonary vascular resistance is relatively high in comparison with the systemic vascular resistance resulting in little blood flow through the fetal lungs. Instead the high pulmonary vascular resistance diverts the blood away from the lungs though the foramen ovale (opening between the left and right atria) and patent ductus artreriosus (the blood vessel connect the pulmonary artery to the aorta) into the low resistance systemic and placental circulation. (2) The second immediate phase occurs within a minute after birth. At birth, the placental circulation is removed and systemic vascular resistance rises leading to an increase in left ventricular and atrial pressures, which help to close the foramen ovale. Upon ventilation, the oxygen tension in the alveolus and arterial blood pressure increases, thereby reducing pulmonary vasoconstriction and subsequently pulmonary vascular resistance to less than systemic resistance. An increase in arterial oxygenation also helps to close the patent ductus arteriosus. The overall result is a shift in blood flow into the lungs and the transformation of the lungs into an air-filled organ essential for the oxygenation of the blood. (3) The fast phase occurs 12 to 24 hours after birth and is characterized by the largest reduction in pulmonary vascular resistance due to the production of endogenous vasodilators, prostacyclin and nitric oxide (NO). Prostacyclin is produced in response to the rhythmic distension of the lungs. Maternal intake of aspirin and non-steroidal anti-inflammatory agents such as indomethacin, which inhibit prostacyclin, may lead to the development of persistent pulmonary hypertension of the newborn (PPHN) in the newborn. NO is released in response to various factors including stretching of the pulmonary vasculature, ventilation, increased oxygenation and clearance of lung fluid. (4) The final phase involves remodeling of the pulmonary vascular musculature, wherein the fully muscularized arteries extending to the terminal bronchioles decrease in thickness within days after delivery (Nair and Bataclan, Saudi Med. J. 25(6):693-699(2004)).

Persistent pulmonary hypertension of the newborn (PPHN) results from the failure of the normal postnatal reduction in pulmonary vascular resistance and is associated with persistent right to left shunts across the fetal channels and resultant hypoxia (Kinsella, et al. J. Pediatr. 126:853-64 (1995)). PPHN is also known as persistent fetal circulation, persistent transitional circulation, persistent pulmonary vascular obstruction or pulmonary vasospasm ((Geggel, et al., Clin. Perinatol. 11:525-549 (1983)) PPHN is typically seen in full term and post term infants or preterm infants (37 to 41 weeks gestational age) and develops within the first 12 to 24 hours afterbirth. Echocardiography provides an accurate diagnosis of PPHN, excludes suspicion of congenital heart disease, defines the pulmonary artery pressure, characterizes the shunt through the ductus arteriosus and foramen ovale, and defines the ventricular outputs (Evans, et al., Arch. Dis. Child (1998)). PPHN occurs in 1 to 6 infants in 1000 live births and is a major cause of morbidity (15-25% neurological handicap) and mortality (2′0-50%) in the term and near-term infant (Pierce, Hospital Medicine 65(7):418-421 (2004)). There are three types of PPHN, including primary PPHN, secondary PPHN, and PPHN associated with hypoplastic lungs.

Primary PPHN presents soon after birth and is characterized by hypoxemia in an infant with clinically and radiologically normal lungs. Primary PPHN may be caused by primary dysfunction in the pulmonary endothelial vasodilating mechanisms. This form of PPHN is usually idiopathic in origin and may be associated with various complications of pregnancy, including maternal diabetes, maternal hypertension, prolonged gestation, maternal ingestion of prostaglandin resulting in premature ductal closure, polycythaemia, fetal anemia and premature ductal closure (Evans, et al., Arch. Dis. Child (1998); Fox, et al., J. Pediatr. 103:505-14 (1983)). Secondary PPHN occurs secondary to a disease in the lung parenchymal tissue. In infants with secondary PPHN, pulmonary vasoconstriction results from hypoxia, acidosis and high ventilatory pressures (Evans, et al., Arch. Dis. Child (1998); Fox, et al., J. Pediatr. 103:505-14 (1983); Evans, et al., Arch. Dis. Child 74: F88-94 (1996)). Secondary PPHN may result from various respiratory disorders including meconium aspiration, group B streptococcal pneumonia, sepsis, respiratory distress syndrome and severe hyaline membrane disease. PPHN associated with hypoplastic lungs is most often seen with diaphragmatic hernia or oligohydramnios. It is characterized by an anatomic reduction in the number of pulmonary capillaries (Nair and Bataclan, Saudi Med. J. 25(6): 693-699 (2004)). This type of PPHN is often included under the classification of secondary PPHN.

The objectives of treatment of PPHN are: (1) to maintain homeostasis, (2) to provide adjuvant therapy and (3) to provide specific therapy to reduce pulmonary pressure. Maintenance of homeostasis involves the correction of factors that predispose to PPHN including hypoxia, acidosis, hypothermia, polycythemia, hypoglycemia, hypocalcemia, and hypomagnesia. Adjuvant therapy consists of sedation, paralysis, treatment of infection, nutritional support and minimal handling of the newborn. Specific therapy is directed at maintaining adequate oxygenation.

One of the primary objectives of treating PPHN is to maintain normal arterial oxygen levels and normal oxygen delivery to the organs of the body. The two most potent natural vasodilators are oxygen and lung inflation. The provision of oxygen will maintain arterial oxygen levels and will act as a pulmonary vasodilator.

Animal data suggests that optimal pulmonary vasodilation occurs with a pO₂ around 120 mmHg. In adults, the normal blood gas values are pH 7.35-7.45, PaCO₂ 35 to 45 mmHg, PaO₂ 75 to 100 mmHg, HCO₃⁻20 to 26 mEq/liter, base excess −2 to +2 mEq/liter and O₂ saturation of 94% to 100%. The normal arterial blood gas values of a neonate are pH 7.35-7.45, PaCO₂ 35 to 45 mmHg, PaO₂ 50 to 70 mmHg (term infant) and 45 to 65 mmHg (preterm infant), HCO₃⁻22 to 26 mEq/liter, base excess −2 to +2 mEq/liter and O₂ saturation of 92% to 94% (Askin, Neonatal Network 16(6):23-29 (1997)). Hematocrit should be maintained at greater than 40%.

Conventional ventilation is the mainstay of respiratory support and necessitates ventilation with high minute volumes of greater than 300 mls/kg. Time cycled pressure limited ventilation (TCPLV) is used at low peak inspiratory pressures in treating PPHN and requires monitoring of PaO₂ and PaCO₂ with a transcutaneous monitor. Hyperventilation helps to promote pulmonary vasodilation. Respiratory alkalosis reduces pulmonary arterial pressures to levels below systemic pressures thereby improving oxygenation and closure of the shunts. The level of pH must be maintained at 7.55 and PaCO₂ between 25 and 30 mmHg. The disadvantages of hyperventilation are potential to cause lung injury and agitation of infant, requiring a need to administer muscle relaxants, such as pancuronium and morphine, and sedation. Hyperventilation (with rates greater than 100 breaths per minute and high peak pressures to achieve a critical PaCO₂) is associated with a high incidence of barotraumas, hearing loss and adverse neurodevelopment outcome. PPHN has been managed successfully without hyperventilation (Marron, et al., Pediatr 90(3):392-6 (1992); Wung, et al., Pediatr. 76(4):488-94 (1985)). Alkalizing agents such as sodium bicarbonate or tris(hydroxy-methyl)aminomethane (THAM) may be useful. Prolonged use and large doses of sodium bicarbonate may be associated with hypernatremia, and THAM infiltration may cause sever injury.

High Frequency Oscillatory Ventilation (HFOV) provides better oxygenation than conventional ventilation in babies with severe hypoxic respiratory failure and has been effective in secondary PPHN (Kinsella, et al., J. Pediatr. 126:853-64 (1995)). High frequency ventilation can be used to effectively manage PPHN and reduce the need for extracorporeal membrane oxygenation (ECMO). However, HIFI has been associated with an increased incidence of intraventricular hemorrhage and periventricular leukomalacia.

Extracorporeal membrane oxygenation (ECMO) is a form of cardiorespiratory support that allows the lungs to rest, wherein gas exchange takes place as the blood is pumped through a membrane oxygenator. ECMO has been shown to significantly reduce mortality in babies with an oxygenation index of greater than 40 and should be considered in infants that do not respond to inhaled nitric oxide and HFOV (Lancet 348:75-82 (1996)). It has been used as rescue therapy for term babies with severe hypoxemic respiratory failure. Infants with severe PPHN, considered to have less than 20% probability of survival, had more than 80% survival rate when treated with ECMO. More than 12,000 newborns have been treated with ECMO as recorded in the Extracorporeal Life Support Organization. ECMO is used most often in infants who experienced meconium aspiration. Complications associated with ECMO treatment include cerebral infarct, hemorrhage and seizures (Kanto, Pediatr. 124(3):335-47 (1994); Wilson, et al., J. Pediatr. Surg. 31(8):1116-23 (1996)).

Inotropic agents dopamine and dobutamine have been administered to infants with PPHN to maintain systemic blood pressure and increase cardiac output.

Vasodilators have been used to reduce pulmonary pressures. Tolazoline and prostacycline are systemic vasodilators and may cause systemic hypotension, but have been shown to produce increases in oxygenation in primary PPHN (Eronen, et al., Pediatr. Cardiol. 18:3-7 (1997)). Treatment with tolazoline, a vasodilator initially used at many centers was associated with only a 60% response rate and high rate of complications, which included systemic hypotension, oliguria, gastrointestinal hemorrhage, duodenal perforation and seizures. Magnesium sulphate, a modulator of vascular contraction and membrane excitability also has effects on muscular relaxation and sedation and caused complications at high doses, including hypotension and respiratory depression (Wu, et al., Pediatr. 96:472-4 (1995)). Nitroprusside, a potent direct acting vasodilator has been used successfully in neonates (Benitz, et al. J. Pediatr. 106(1):102-10 (1985)). Dipyramidole, which inhibits phosphodiesterase 5, an enzyme that inactivates cGMP, was associated with unacceptable systemic hemodynamic disturbances in patients with PPHN (Dukarm, et al., Pediatr. Res. 44(4):831-7 (1998). Treatment with the combination of parenteral vasodilators such as nitroprusside and dipyridamole are under evaluation (Benitz, et al., J. Perinatol. 16(6):443-8 (1996); Thebaud, et al., Intensive Care Med. 25(3):300-3 (1999)). Treatment of PPHN with adenosine triphosphate produced a response in 5 out of 6 infants with PPHN without any adverse side effects such as bradycardia, hypotension, or prolonged bleeding time (Patole, et al., 74(5):345-50 (1998)).

Treatment with inhaled nitric oxide (NO) in term newborns was first published in 1992 and has since been evaluated with respect to dosing, disease-related response and toxicity (I-NO/PPHN study group. Pediatr 101:325-34 (1998); Mercier, et al, Eur. J. Pediatr. 101:325-34 (1998); Mercier, et al., Eur. J. Pediatr. 157(9):747-52 (1998); George, et al, J. Pediatr. 132:731-4 (1998); Hallman, et al., J. Pediatr. 132:827-9 (1998). NO is the vasodilator of choice in term infants with PPHN (Finer, Arch. Dis. Child 77:F81-4 (1997). Studies have shown that NO significantly improves oxygenation and reduces the need for rescue with ECMO (Roberts, et al., N. Engl. J. Med. 336:605-10 (1997); The Neonatal Inhaled Nitric Oxide Study Group, N. Engl. J. Med. 336:597-604 (1997)). The optimal dose is probably between 10 to 40 ppm. When NO reaches 80 ppm, the blood concentrations of methemoglobin and nitrogen dioxide increase (1-NO/PPHN study group, Pediatr. 101:325-34 (1998)). NO is a more specific vasodilator and has superseded tolazoline and prostacyclin. The response to NO depends on the underlying pathophysiology and NO has been shown to effective in treating primary PPHN. However, NO was shown to be ineffective in some patients with secondary PPHN and HFOV was required to supplement the response to NO by improving lung inflation and minimizing regional atelectasis (Kinsella, et al., J. Pediatr. 131:55-62 (1997). NO is recommended in infants with severe hypoxic respiratory failure characterized by inability to maintain a PaO₂ above 80 mmHg despite maximal respiratory support and in ventilated infants with a significant (>50%) oxygen requirement and echocardiographic evidence of pulmonary artery pressure close to or above systemic pressure with poor cardiac output (<150 mls/kg/min).

Thus, there remains a need for a consistently effective and specific agent for treating PPHN without causing severe adverse side effects. The present invention is directed to addressing such a need.

### SUMMARY OF THE INVENTION

In general, the invention describes a therapeutic intervention of Persistent Pulmonary Hypertension of the Newborn (PPHN). In one embodiment, the invention provides a method for treating a subject having below normal arterial oxygen pressure (PaO₂) comprising administering to said subject a composition comprising tetrahydrobiopterin (BH4) or a precursor or derivative thereof, wherein the administration of BH4 is effective to increasing PaO₂ of said subject as compared to said PaO₂ in the absence of said BH4. In a preferred embodiment, the invention provides a method for treating a subject diagnosed as having a Persistent Pulmonary Hypertension of the Newborn (PPHN).

In one aspect, the present invention is directed to methods and compositions for the treatment of subjects with primary PPHN, wherein subjects exhibit hypoxemia with clinically and radiologically normal lungs. Primary PPHN may be caused by primary dysfunction in the pulmonary endothelial vasodilating mechanisms and associated with various conditions, disorders and diseases including but not limited to complications of pregnancy, including maternal diabetes, maternal hypertension, prolonged gestation, and maternal indomethacin, and also polycythaemia, fetal anemia and premature ductal closure. In another aspect, the invention is directed to methods and compositions for the treatment of subjects with secondary PPHN occurring secondary to a disease in the lung parenchymal tissue. Secondary PPHN may result from pulmonary vasoconstriction from hypoxia, acidosis and high ventilatory pressures. Secondary PPHN may be associated with various respiratory disorders including but not limited to meconium aspiration, pneumonia and severe hyaline membrane disease. In a further aspect, the invention is directed to methods and compositions for the treatment of subjects with PPHN associated with diaphragmatic hernia and other forms of pulmonary hypoplasia.

In preferred embodiments, subjects are preterm infants (less than 37 weeks gestational age), full (gestational age between 37 and 42 completed weeks), or post term (born 2 weeks or more than the usual 9 months or 280 days of gestation) infants and exhibit: (1) a PaO₂ of less than 50 mmHg and/or greater than 15 mmHg PaO₂ difference between preductal and postductal arterial blood gases when placed on 100% O₂ (hyperoxia test); or (2) a PaO₂ of 100 mmHg when infant is hyperinflated with a manual resuscitator in 100% O₂ until PaCO₂ reaches 20-25 mmHg (hyperoxia-hyperventilation test); or (3) a PaO₂ of less than 100 mmHg when subjected to the hyperoxia-hyperventilation test and a normal echo on the echocardiogram or (4) a right ventricular ratio of greater than 0.50 and left ventricular ratio of greater than 0.38. In most preferred embodiments, infants are diagnosed with PPHN.

The invention contemplates methods of treating a subject having PPHN, comprising administering a BH4 composition to said subject in an amount effective to produce an increase in PaO₂. In preferred embodiments, the administering of BH4 increases PaO₂ to greater than 45 mmHg in infants with PPHN. In more preferred embodiments, the administering of BH4 increases PaO₂ to between about 45 and 120 mmHg, and more preferably between 50 to 100 mmHg in infants with PPHN.

BH4 is administered in an amount of between about 0.1 mg/kg to about 30 mg/kg. BH4 may be administered in a single daily dose or in multiple doses on a daily basis. In some embodiments, the BH4 therapy is not continuous, but rather BH4 is administered on a daily basis until PaO₂ is maintained at greater than 45 mmHg, more preferably between about 45 and 120 mmHg and most preferably between 50 to 100 mmHg. The level of PaCO₂ should be maintained at normal to low levels in the range of 25 to 45 mmHg and most preferably 35 to 45 mmHg. The pH of arterial blood should be between pH 7.35 and 7.55 and most preferably between pH 7.35 and pH 7.45. Oxygen saturation should be maintained between 92% and 100%, more preferably between 94% and 99%, and most preferably at greater than 95%. Preferably, wherein the PaO₂ of the subject is monitored on a continuous basis and the BH4 is administered when a 10 mmHg or 20% increase in PaO₂ is observed.

Also contemplated is a composition comprising a stabilized, crystallized form of BH4 that is stable at room temperature for more than 8 hours and a pharmaceutically acceptable carrier, diluent or excipient. In other embodiments, the BH4 composition is part of an infant formula. Preferably, the BH4 being administered is a stabilized crystallized form of BH4 that has greater stability than non-crystallized stabilized BH4. More preferably, the stabilized crystallized form of BH4 comprises at least 99.5% pure 6R BH4. Precursors such as dihydrobiopterin (BH2), and sepiapterin also may be administered. BH4 may be administered orally.

BH4 may be administered intramuscularly, subcutaneously, or intravenously, via intrapulmonary administration either alone or in combination with other therapeutic agents or interventions currently used to treat PPHN including but not limited to agents and intervention used to maintain homeostasis, adjuvant therapy and specific therapy to provide adequate oxygenation such as vasodilators. Such therapeutic agents and interventions used to maintain homeostasis such as to correct factors predisposing PPHN including hypoxia, acidosis, hypothermia, polycythemia, hypoglycemia, hypocalcemia, and hypomagnesia. Such adjuvant therapy includes but is not limited to agents and intervention that induce sedation and paralysis, treat infection, and provide nutritional support. Such specific therapy directed to improving oxygenation and thereby reducing pulmonary resistance may include high frequency ventilation, extracorporeal membrane oxygenation (ECMO), and vasodilators, including but not limited to tolazoline, magnesium sulphate, nitropresside, prostacyclin, dipyramidole, adenosine triphosphate, inhaled NO, and factors associated with enhancing the activity of nitric oxide synthase.

The present invention contemplates administering one or more of crystal form of BH4 selected from the group consisting of crystal polymorph form A, crystal polymorph form B, crystal polymorph form F, crystal polymorph form J, crystal polymorph form K, crystal hydrate form C, crystal hydrate form D, crystal hydrate form E, crystal hydrate form H, crystal hydrate form O, solvate crystal form G, solvate crystal form I, solvate crystal form L, solvate crystal form M, solvate crystal form N, and combinations thereof.

In other embodiments, BH4 may be administered optionally and concurrently with folates, including folate precursors, folic acids, and folate derivatives. Such folates include but are not limited to tetrahydrofolate is 5-formyl-(6S)-tetrahydrofolic acid and salts thereof, 5-methyl-(6S)-tetrahydrofolic acid and salts thereof, 5,10-methylene-(6R)-tetrahydrofolic acid and salts thereof, 5,10-methenyl-(6R)-tetrahydrofolic acid and salts thereof, 10-formyl-(6R)-tetrahydrofolic acid, 5-formimino-(6S)-tetrahydrofolic acid salts thereof, (6S)-tetrahydrofolic acid and salts thereof, and combinations of the foregoing. In a further embodiment, BH4 may be administered optionally and concurrently with arginine.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1. Powder X-ray Diffraction Pattern of (6R)-L-erythro-tetrahydrobiopterin dihydrochloride Form B.

FIG. 2. Powder X-ray Diffraction Pattern of (6R)-L-erythro-tetrahydrobiopterin dihydrochloride Form A.

FIG. 3. Powder X-ray Diffraction Pattern of (6R)-L-erythro-tetrahydrobiopterin dihydrochloride Form F.

FIG. 4. Powder X-ray Diffraction Pattern of (6R)-L-erythro-tetrahydrobiopterin dihydrochloride Form J.

FIG. 5. Powder X-ray Diffraction Pattern of (6R)-L-erythro-tetrahydrobiopterin dihydrochloride Form K.

FIG. 6. Powder X-ray Diffraction Pattern of (6R)-L-erythro-tetrahydrobiopterin dihydrochloride Form C.

FIG. 7. Powder X-ray Diffraction Pattern of (6R)-L-erythro-tetrahydrobiopterin dihydrochloride Form D.

FIG. 8. Powder X-ray Diffraction Pattern of (6R)-L-erythro-tetrahydrobiopterin dihydrochloride Form E.

FIG. 9. Powder X-ray Diffraction Pattern of (6R)-L-erythro-tetrahydrobiopterin dihydrochloride Form H.

FIG. 10. Powder X-ray Diffraction Pattern of (6R)-L-erythro-tetrahydrobiopterin dihydrochloride Form O.

FIG. 11. Powder X-ray Diffraction Pattern of (6R)-L-erythro-tetrahydrobiopterin dihydrochloride Form G.

FIG. 12. Powder X-ray Diffraction Pattern of (6R)-L-erythro-tetrahydrobiopterin dihydrochloride Form I.

FIG. 13. Powder X-ray Diffraction Pattern of (6R)-L-erythro-tetrahydrobiopterin dihydrochloride Form L.

FIG. 14. Powder X-ray Diffraction Pattern of (6R)-L-erythro-tetrahydrobiopterin dihydrochloride Form M.

FIG. 15. Powder X-ray Diffraction Pattern of (6R)-L-erythro-tetrahydrobiopterin dihydrochloride Form N.

FIG. 16. Mean blood Phe level 3 and 7 days after multiple daily BH4 doses of 10 and 20 mg/kg in PKU Patients (N=20).

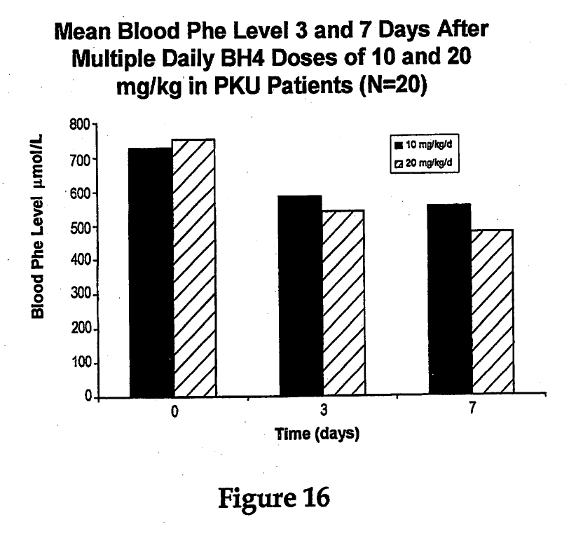

FIG. 17. Individual blood Phe in 12 adults with PKU on 10 mg/kg BH4 administered daily.

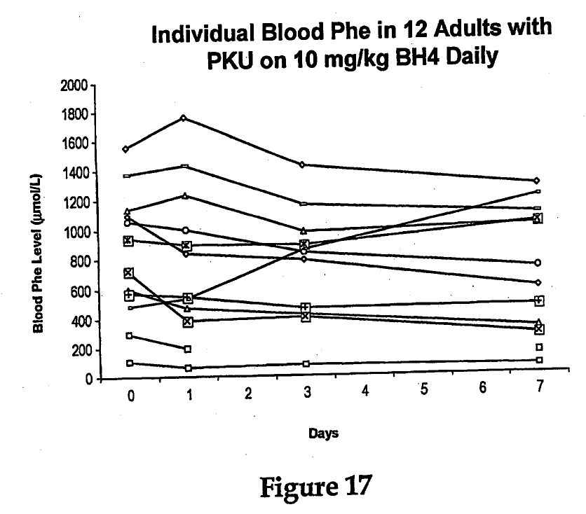

FIG. 18. Individual blood Phe in 12 adults with PKU on 20 mg/kg BH4 administered daily.

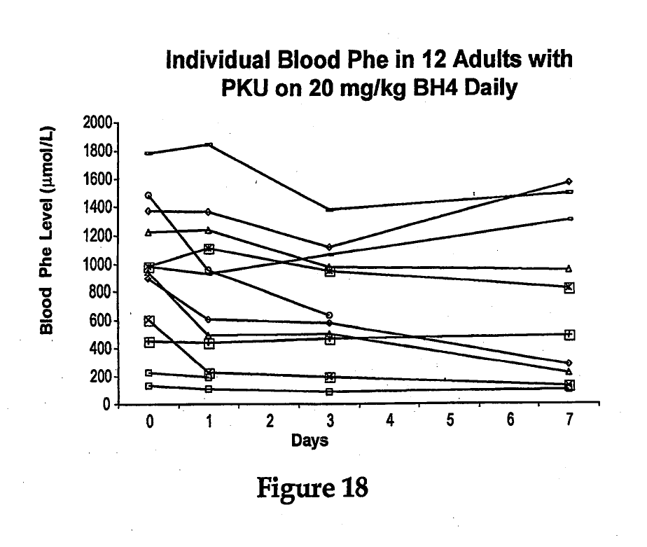

FIG. 19. Individual blood Phe in 8 children with PKU on 10 mg/kg BH4 administered daily.

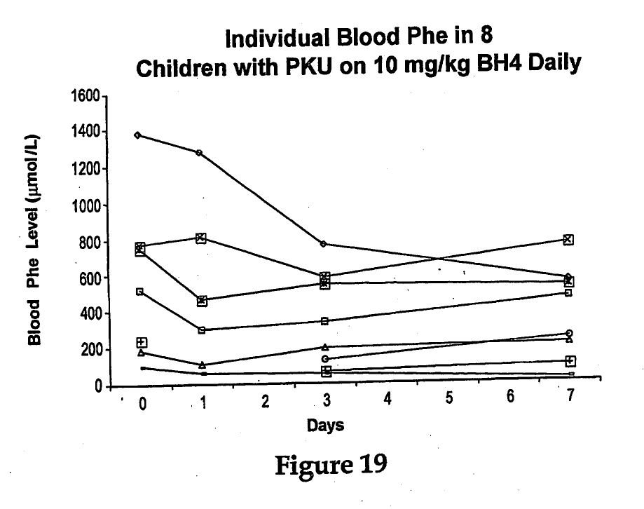

FIG. 20. Individual blood Phe in 8 children with PKU on 20 mg/kg BH4 administered daily.

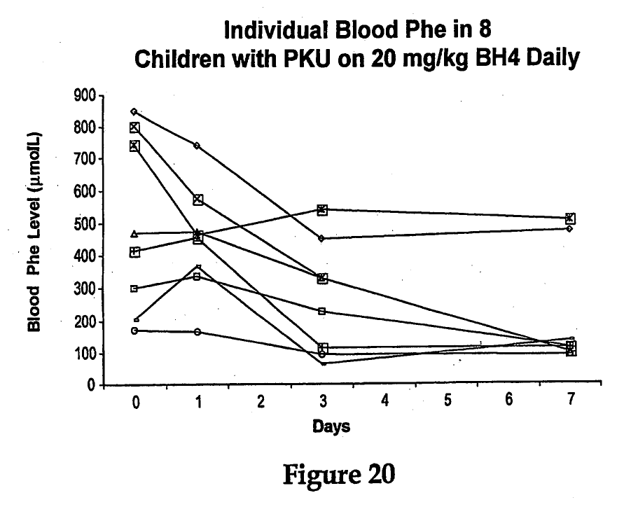

### DESCRIPTION OF THE PREFERRED EMBODIMENTS

### Persistant Pulmonary Hypertension of the Newborn (PPHN)

As described above, Persistent Pulmonary Hypertension of the Newborn (PPHN) results from the failure of the normal postnatal reduction in pulmonary vascular resistance and is associated with persistent right to left shunts across the fetal channels and resultant hypoxia (Kinsella, et al. J. Pediatr. 126:853-64 (1995)). PPHN most often occurs in full term (gestational age between 37 and 42 completed weeks) and post term infants (born two weeks or more after the usual 9 months or 280 days of gestation) and progresses over the first 12 to 24 hours after birth. PPHN is most accurately diagnosed by echocardiography, which can rule out congenital heart disease, define the pulmonary artery pressure, characterize the shunt through the ductus arteriosus and foramen ovale, and define the ventricular outputs (Evans, et al., Arch. Dis. Child (1998)). PPHN may be classified as primary, secondary or associated with hypoplastic lungs. Infants with primary PPHN have clinically and radiologically normal lungs, whereas secondary PPHN is associated with disease of the lung parenchymal tissue. Primary PPHN may be caused by primary dysfunction in the pulmonary endothelial vasodilating mechanisms, whereas pulmonary vasoconstriction in secondary PPHN results from hypoxia, acidosis and high ventilatory pressures (Evans, et al., Arch. Dis. Child (1998); Fox, et al., J. Pediatr. 103:505-14 (1983); Evans, et al., Arch. Dis. Child 74: F88-94 (1996)). Primary PPHN is usually idiopathic in origin and may be associated with various complications of pregnancy, including maternal diabetes, maternal hypertension, prolonged gestation, maternal ingestion of prostaglandin resulting in premature ductal closure, polycythaemia, fetal anemia and premature ductal closure (Evans, et al., Arch. Dis. Child (1998); Fox, et al., J. Pediatr. 103:505-14 (1983)). Secondary PPHN may result from various respiratory disorders including meconium aspiration, group B streptococcal pneumonia, sepsis, respiratory distress syndrome and severe hyaline membrane disease. PPHN associated with hypoplastic lungs is characterized by an anatomic reduction in the number of pulmonary capillaries (Nair and Bataclan, Saudi Med. J. 25(6): 693-699 (2004)).

### Clinical Diagnosis

Infants with PPHN may have Apgar scores of 5 or less at 1 and 5 minutes and cyanosis may be present at birth or gradually worsen within the first 12 to 24 hours. Symptoms of primary PPHN include cyanosis with some degree of respiratory distress in the early postnatal period, resemblance to cyanotic congenital heart disease, clear or minimally opacified lung fields on X-ray, variable degree of hypoxia and normal or low pCO₂. The symptoms of secondary PPHN include primarily respiratory distress and parenchymal lung opacity on X-ray. In both types of PPHN, patients may exhibit prominent precordial impulse, low parasternal murmur of tricuspid incompetence and a large cardiac shadow on X-ray.

### Hyperoxia Test

An infant suspected of having PPHN is placed on 100% oxyhood for 10 minutes. If PaO₂ is greater than 100 mmHg, parenchymal lung disease is suspected. If PaO₂ is between 50 and 100 mmHg, either parenchymal lung, disease or cardiovascular disease is suspected. If PaO₂ is less than 50 mmHg, a fixed right to left shunt is suspected and suggests either cyanotic congenital heart disease or PPHN.

### Comparison of Preductal and Postductal Arterial PaO₂

In the case of suspected right to left shunt, preductal and postductal arterial blood gases are determined in an infant on 100% O₂. A difference in PaO₂ of greater than 15 mmHg confirms ductal shunting. The preductal measurement can be taken from the right radial or temporal artery and the postductal from the umbilical cord or left foot. The preductal and postductal arterial oxygen pressures can be monitored continuously to assess improvement in shunting.

### Hyperoxia-Hyperventilation Test

The infant is hyperinflated with a manual resuscitator and 100% O₂ until PaCO₂ reaches between 20 and 25 mmHg. If PaO₂ is 100 mmHg with hyperinflation, PPHN is suspected. If PaO₂ is less than 100 mmHg with hyperinflation, either congenital heart disease or PPHN may be suspected and subsequent echocardiography can be used to provide a definitive diagnosis of congenital heart disease (echo is abnormal) or PPHN (echo is normal).

### Echocardiographic Diagnosis

Echocardiographic diagnosis provides an accurate diagnosis of PPHN and can exclude congenital heart disease. Echocardiography defines the pulmonary artery pressure using tricuspid incompetence or ductal shunt velocities, the presence, degree and direction of shunt through the duct and foramen ovale and ventricular outputs. The ratio of pre-ejection period (PEP) to ejection time (ET) provides an evaluation of left and right ventricle performance. PPHN is associated with a prolonged right ventricle PEP/ET ratio due to increased pulmonary artery pressure and increased pulmonary vascular resistance. PPHN can be identified early if right and left ventricular PET/ET ratios are measured soon after birth. Infants with a right ventricular ration of greater than 0.5 and left ventricular ration of greater than 0.38 developed PPHN within 10 to 30 hours after birth.

### Cardiac Catheterization

Cardiac catheterization has been used to diagnose infants with PPHN by monitoring pulmonary artery pressures but is not recommend because it is traumatic and has been replaced by less invasive measures.

It is contemplated that the arterial oxygen pressures of the patients will be monitored at convenient intervals (e.g., continuously, daily, every other day or weekly) throughout the time course of the therapeutic regimen. By monitoring the arterial oxygen pressures with such regularity, the clinician will be able to assess the efficacy of the treatment and adjust the BH4 requirements accordingly.

Role of Nitric oxide (NO) in Vasodilation

The pulmonary endothelium plays a significant role in the adaptation and regulation of vascular tone. Nitric oxide is constitutively produced by vascular endothelial cells where it plays a key physiological role in the regulation of blood pressure and vascular tone. Deficient nitric oxide bioactivity is involved in the pathogenesis of vascular dysfunctions, including coronary artery disease, atherosclerosis of any arteries, including coronary, carotid, cerebral, or peripheral vascular arteries, ischemia-reperfusion injury, hypertension, diabetes, diabetic vasculopathy, cardiovascular disease, peripheral vascular disease, or neurodegenerative conditions stemming from ischemia and/or inflammation, such as stroke, and that such pathogenesis includes damaged endothelium, insufficient oxygen flow to organs and tissues, elevated systemic vascular resistance (high blood pressure), vascular smooth muscle proliferation, progression of vascular stenosis (narrowing) and inflammation. Within the vascular endothelium, the smooth muscle relaxant, endothelial nitric oxide (eNO), is synthesized from 1-arginine by nitric oxide synthase (NOS). eNO reduces the resting pulmonary vascular tone and thereby reduces pulmonary pressure. More specifically eNO relaxes smooth muscle cells by activating guanylate cyclase, thereby increasing cyclic guanosine 3′,5′ cyclic monophosphate (cGMP) concentrations and triggering a series of events leading to relaxation of arterial smooth muscle. (Gao et al., Circulation Research 76:559-565 (1995), incorporated herein in its entirety by reference) The cGMP signal transduction mechanism is controlled by the phosphodiesterases, which metabolize 3′,5′ cyclic nucleotides.

Other studies have found that NO plays a role in pulmonary vasoconstriction (Ogata, et al., Am J Physiol 262:H691-H697 (1992); Carville et al., J Cardiovasc Pharmacol 22(6):889-896 (1993); Kovitz et al., Am J Physiol 265:H139-H148 (1993); and Villamor et al., Biol Neonate 72(1):62-70 (1997), each of which is incorporated herein in its entirety by reference). Some studies have also shown a role for the superoxide anion (O₂⁻) in modulating NO bioavailability, wherein O₂⁻ reacts with NO to form ONOO⁻ (peroxynitrite) and prevents the vasodilating activity of NO. Here, superoxide dismutase (SOD), the scavenging enzyme of O₂⁻, is important in maintaining the bioavailability of NO, protecting it from the destructive action of endogenously produced O₂⁻. (Villamor et al., Pediatric Research 54:372-381 (2003); Wedgwood et al., Am J Physiol Lung Cell Mol Physiol 288(3):L480-L487 (2005) and Wedgwood et al., Am J Physiol Lung Cell Mol Physiol 289(4):L660-L666 (2005); incorporated herein by reference in their entireties) Other studies have focused on PDE inhibitors as a means of affecting NO levels in the newborn to control PPHN. (Bassler et al. Biol Neonate 89(1):1-5 (2005); Juliana et al. Eur J Pediatr 164(10):626-629 (2005); incorporated herein by reference in their entireties) Still other approaches have been to study modulators of guanylate cyclase (Deruell et al., Am J Physiol Lung Cell Mol Physiol 289(5):L798-L806 (2005); incorporated herein by reference in its entirety), which, as described above, is a downstream effector of eNO activity.

PPHN may result from an imbalance between vasoconstricting and vasodilating factors. One mechanism may be the abnormal responsiveness of the pulmonary vasculature to hypoxia resulting in an inability to relax. Another mechanism may be alterations in vasoactive mediator levels. Vosatka, et al. (Biol. Neonate 66(2-3):65-70 (1994)) reported arginine deficiency in infants with PPHN and the metabolism of arginine may play a role in differing responses of the hypoxic newborn to NO. The concentration of endothelin-1, a vasoconstrictor, is increased in neonates with PPHN and modulated by inhaled NO, whereas cGMP plasma concentrations were reduced in infants with PPHN (Rosenberg, et al., J. Pediatr. 123(1):109-14 (1993); Christou, et al. J. Pediatr. 130:603-11 (1997); Kuo and Chen, Biol. Neonate 76:228-34 (1999)). Platelet activating factor, an endogenous phospholipid mediator that causes pulmonary hypertension in an animal model was increased in neonates with PPHN (Caplan, et al., Am. Rev. Resp. Dis. 142(6 pt 1):1258-62 (1990)). One pilot study showed that endogenous NOS mRNA was detected in all normal term infants but was notably absent in the majority of infants with PPHN (Villanueva, et al., Pediatr. Res. 44(3):338-43 (1998)). It is not clear whether the decreased eNOS transcript is a cause of PPHN or is a result of intrapartum stress. One study showed that endothelial nitric oxide synthase (eNOS), a major source of the potent vasodilator and antiproliferative product eNO, is downregulated in the adult Fawn-Hooded rat, a genetic strain that serves as a model of primary pulmonary tension in adult rats (Tyler, et al., Am. J. Physiol. Lung Cell Mol. Physiol. 276:L297-L303 (1999)). Pulmonary adenosine levels were reduced in fetal as compared to newborn lambs and in patients with pulmonary hypertension (Konduri, et al., Pediatrics 97:295-300 (1996); Saadjian, et al., Am. J. Cardiol. 85:858-63 (2000)).

Unlike systemic vasodilators such as tolazoline and prostacyclin, inhaled NO selectively dilates the pulmonary vasculature secondary to rapid inactivation of NO by hemoglobin. More importantly, NO is thought to target the underlying pathophysiology of PPHN. Studies have shown that lambs with PPHN showed a down-regulation of NO production, characterized by impaired endothelium-dependent pulmonary vasodilation, decreased NO synthase activity, and decreased endothelial NO synthase gene expression. Human infants with PPHN have also demonstrated a decrease in urinary NO metabolites, suggesting a similar down regulation in NO production. (Ahman, et al., J. Clin. Invest. 83:1849-1858 (1989); Shaul, et al., Am. J. Physiol. 272(5 Pt 1):L1005-11012 (1997)). The basis for the inconsistent response to NO is uncertain but may be due to several factors and the underlying pathophysiology of PPHN. Studies in lambs with PPHN and lung maldevelopment showed that soluble guanylate cyclase expression was decreased and phosphodiesterase 5 expression was increased, changes that would reduce the levels of cyclic GMP, the second messenger which mediates NO-induced relaxation. Thus, the low levels of cGMP may be the basis for a lack of response to NO (Tzao, et al., Pediatr. Pulmonol 31:97-105 (2001)). However, combined therapy with inhaled NO and phosphodiesterase inhibitors have exhibited only intermittent success (Kinsella, et al. Lancet 346:647-648 (1995)).

A current area of interest in the treatment of PPHN is the role of cyclic GMP, the second messenger in muscle relaxation (Steinhorn, et al., Perinatol. 21(5):393-408 (1997)). Sildenafil is a selective inhibitor of the enzyme, phosphodiesterase type 5 (PDE5) that inactivates cGMP and may be useful in amplifying the NO signaling cascade (Jackson, et al., Am. J. Cardiol. 83:13C-20C (1999); Dukarm, et al., Am. J. Respir. Crit. Care Med. 160:858-65 (1999); Weimann, et al., Anesthesiology, 92::1702-12 (2000); Wallis, et al., Am. J. Cardiol. 83:3C-12C (1999): Wallace and Tom Anesth. Analg. 90:840-6 (2000), Atz and Wessel, Anesthesiology 91:301-10 (1999); Abrams, et al., Heart 84:e4-5 (2000)). United States Patent Application Publication No. 2004/0127449A1, herein incorporated by reference, describes a gene therapy method for inducing pulmonary vasodilation by introducing the nitric oxide synthase gene into the lungs without affecting systemic blood pressure or cardiac index.

Tetrahydrobiopterin (BH4) is a cofactor in the biosynthesis of NO with NOS, and when administered to patients with NOS dysfunction such as PPHN, BH4 may prevent or treat these diseases by activating the functions of NOS, increasing NO production and suppressing the production of active oxygen species to improve disorders of vascular endothelial cells. The use of tetrahydrobiopterin and/or its derivatives in the treatment of pulmonary hypertension in general has been described in European Patent No. 0908182B1 and International Application Publication Nos. WO 2004/017955 and WO 2002/17898, the disclosures of which are herein incorporated by reference. However, pulmonary hypertension differs from PPHN with respect to epidemiology (affects children and adults), pathophysiology (caused by congenital heart defects, connective tissue disease, certain medications, HIV infection, blood clots, liver disease, unknown causes), clinical presentation (unusual fatigue, shortness of breath, chest pain, loss of consciousness, ankle swelling), diagnosis (chest X-ray, autoantibody blood tests, liver function tests, heart catheterization, CAT scans) and treatment (prostacyclin, calcium channel blockers, bosentan, anticoagulants, digoxin, diuretics, thromboendarterectomy, and lung transplantation). Unlike PPHN, where prostacyclin produces nonspecific effects such as systemic hypotension, prostacyclin is one of the most effective treatments in pulmonary hypertension. The provision of oxygen is an essential aspect of treating PPHN but is only provided as a supplemental therapy to provide relief and comfort in some patients with pulmonary hypertension (Nauser and Stites, Am. Fam. Physician 63:1789-98, 1800 (2001); Benistry, Circulation 106:e192-4 (2002)).

The present invention for the first time describes a pharmaceutical intervention of PPHN based on the administration of BH4. It is further contemplated that any type of BH4, in a stabilized or other form may be used to treat that patient population comprising subjects with various forms of PPHN, including primary PPHN, secondary PPHN, and PPHN associated with pulmonary hypoplasia. Such BH4-based compositions may be administered alone or in combination with any other therapeutic agent and/or intervention (e.g., ventilation) that is commonly used for the treatment of PPHN.

Certain embodiments of the present invention are directed to treating PPHN by administering to the subject a composition comprising BH4 or a precursor or derivative thereof alone or in combinations with conventional PPHN treatment, wherein the administration of BH4 alone or in combination with conventional PPHN therapy is effective to increase PaO₂ of said subject as compared to said concentration in the absence of BH4 alone or in combination with conventional PPHN therapy.

One embodiment of the invention entails administering a BH4 composition to any individual with a lower than normal PaO₂ in an amount effective to increase such PaO₂ to normal values. In a preferred embodiment, such individual is diagnosed with PPHN. In a more preferred embodiment, such individual is an infant, wherein such infant may be preterm i.e. less than 37 weeks gestational age, full term between 37 and 42 weeks gestational age, or post term born two or more weeks after the usual 9 months or 280 days of gestation. In a most preferred embodiment, such preterm, full or post term infant is characterized by (1) a PaO₂ of less than 50 mmHg and/or greater than 15 mmHg PaO₂ difference between preductal and postductal arterial blood gases when placed on 100% O₂ (hyperoxia test); or (2) a PaO₂ of 100 mmHg when said infant is hyperinflated with a manual resuscitator in 100% O₂ until PaCO₂ reaches between 20 and 25 mmHg (hyperoxia-hyperventilation test); or (3) a PaO₂ of less than 100 mmHg when subjected to the hyperoxia-hyperventilation test and a normal echo on the echocardiogram or (4) a right ventricular ratio of greater than 0.50 and left ventricular ratio of greater than 0.38.

Animal data suggests that optimal pulmonary vasodilation occurs with a pO₂ around 120 mmHg. In adults, the normal blood gas values are pH 7.35-7.45, PaCO₂ 35 to 45 mmHg, PaO₂ 75 to 100 mmHg, HCO₃− 20 to 26 mEq/liter, base excess −2 to +2 mEq/liter and O₂ saturation of 94% to 100%. The normal arterial blood gas values of a neonate are pH 7.35-7.45, PaCO₂ 35 to 45 mmHg, PaO₂ 50 to 70 mmHg (term infant) and 45 to 65 mmHg (preterm infant), HCO3-22 to 26 mEq/liter, base excess −2 to +2 mEq/liter and O₂ saturation of 92 to 94% (Askin, Neonatal Network 16(6):23-29 (1997). Thus, pO₂ should ideally be maintained at greater than 45 mmHg, more preferably between 45 and 120 mmHg, and most preferably between 50 to 100 mmHg. The level of pCO₂ should be maintained at normal to low levels in the range of 25 to 45 mmHg and most preferably 35 to 45 mmHg. The pH of arterial blood should be between pH 7.35 and 7.55 and most preferably between pH 7.35 and pH 7.45. Oxygen saturation should be maintained between 92% and 100%, more preferably between 94% and 99%, and most preferably greater than 95%. Hematocrit should be maintained at greater than 40%.

The invention contemplates administering the stabilized BH4 compositions described herein to infants diagnosed with PPHN or characterized by (1) a PaO₂ of less than 50 mmHg and/or greater than 15 mmHg PaO₂ difference between preductal and postductal arterial blood gases when placed on 100% O₂ (hyperoxia test); or (2) a PaO₂ of 100 mmHg when infant is hyperinflated with a manual resuscitator in 100% O₂ until PaCO₂ reaches 20-25 mmHg (hyperoxia-hyperventilation test); or (3) a PaO₂ of less than 100 mmHg when subjected to the hyperoxia-hyperventilation test and a normal echo on the echocardiogram or (4) a right ventricular ratio of greater than 0.50 and left ventricular ratio of greater than 0.38, in an amount effective to increase PaO₂ to greater than 45 mmHg, more preferably between about 45 and 120 mmHg, most preferably between 50 to 100 mmHg.

Those of skill in the art would understand that the invention contemplates treating infants with arterial oxygen pressures of less than 45 mmHg PaO₂ with BH4 to produce increases in arterial oxygen pressure to greater than 45 mmHg, preferably between 45 and 120 mmHg, and most preferably between 50 to 100 mmHg. Further, any increase in arterial oxygen pressures over 10 mmHg or 20% of the initial arterial oxygen pressure will be considered a therapeutic outcome for the therapeutic regimens for the infants.

In preferred embodiments the arterial oxygen pressure of the PPHN patient being treated is increased from any amount of unrestricted pulmonary pressure that is less than PaO₂ 45 mm Hg to any pulmonary pressure that is greater than PaO₂ 60 mmHg. Of course, even if the treatment with the BH4 produces a lesser increase in arterial oxygen pressure, e.g., to a level of between PaO₂ 45 mm Hg to about PaO₂ 55 mmHg, this will be viewed as a clinically useful outcome of the therapy because patients that have a systemic oxygen pressure in this range can manage the disease by reducing dependence on agents and interventions and the potential for adverse side effects.

### Combination Therapy

The present invention further contemplates the therapeutic intervention of various types of PPHN by administration of BH4 alone or in combination with an agent or intervention commonly used to treat PPHN. It should be understood that the BH4 therapies may be combined with conventional agents or interventions to treat PPHN to effect the therapeutic increase in arterial oxygen pressures in such infants. As described above, treatment of PPHN is directed at maintaining homeostasis, providing adjuvant therapy and providing specific therapy to reduce pulmonary pressure. Homeostasis is maintained by correcting factors that predispose to PPHN including hypoxia, acidosis, hypothermia, polycythemia, hypoglycemia, hypocalcemia, and hypomagnesia. Adjuvant therapy consists of administering agents or interventions that reduce activity of the infant such as sedation, paralysis and minimal handling of the newborn, as well as sustain the health of the infant such as treatment of infection and nutritional support. Specific therapy is directed at maintaining normal arterial oxygen levels and normal oxygen delivery to the organs of the body. Oxygen is provided to maintain arterial oxygen levels and stimulate pulmonary vasodilation. The conventional agents and interventions currently used to treat PPHN have been discussed above. Some of the conventional interventions used to manage or treat PPHN include time cycled pressure limited ventilation (TCPLV), hyperventilation, induction of respiratory alkalosis, High Frequency Oscillatory Ventilation (HFOV), and Extracorporeal membrane oxygenation (ECMO). Some adjuvant agents discussed previously include pancuronium to induce muscle relaxation, morphine and other narcotic drugs to induce sedation, and inotropic agents such dobutamine and dopamine to increase cardiac output and maintain systemic pressure. Various agents have been used to induce vasodilation have also been discussed in detail previously. Conventional vasodilator agents used to manage and/or treat PPHN include inhaled nitric oxide, tolazoline and prostacycline, magnesium sulphate, nitropresside, dipyramidole, and adenosine triphosphate.

The BH4 to be administered alone or in combination with therapeutic agents and interventions to manage and/or treat PPHN, need not necessarily be a stabilized BH4 composition described herein. Those of skill in the art are aware of methods of producing a BH4 composition that is unstable at room temperature and in light. While therapies using such a composition are hindered by the instability of the BH4 composition, its use is still contemplated in certain combination therapies where patients suffering from PPHN are treated with a course of BH4 treatment and conventional PPHN therapy.

The methods and compositions for producing such a stabilized BH4 compositions are described in further detail in Example 2. The stabilized BH4 compositions of the present invention comprise BH4 crystals that are stable at room temperature for longer than 8 hours. The methods and compositions of the present invention contemplate pharmaceutical compositions of the stabilized BH4 alone that may be delivered through any conventional route of administration, including but not limited to oral, intramuscular injection, subcutaneous injection, intravenous injection and the like. The compositions of the present invention may further comprise BH4 compositions in combination with an antioxidant that aids in prolonging the stability of the BH4 composition.

### BH4 Compositions for Use in the Treatment

The present section provides a discussion of the compositions that may be used in the treatments contemplated herein.

U.S. Pat. Nos. 5,698,408; 2,601,215; 3,505,329; 4,540,783; 4,550,109; 4,587,340; 4,595,752; 4,649,197; 4,665,182; 4,701,455; 4,713,454; 4,937,342; 5,037,981; 5,198,547; 5,350,851; 5,401,844; 5,698,408 and Canadian application CA 2420374 (each incorporated herein by reference) each describe methods of making dihydrobiopterins, BH4 and derivative thereof that may be used as compositions for the present invention. Any such methods may be used to produce BH4 compositions for use in the therapeutic methods of the present invention.

U.S. Pat. Nos. 4,752,573; 4,758,571; 4,774,244; 4,920,122; 5,753,656; 5,922,713; 5,874,433; 5,945,452; 6,274,581; 6,410,535; 6,441,038; 6,544,994; and U.S. Patent Publications US 20020187958; US 20020106645; US 2002/0076782; US 20030032616(each incorporated herein by reference) each describe methods of administering BH4 compositions for various treatments. Each of those patents is incorporated herein by reference as providing a general teaching of methods of administering BH4 compositions known to those of skill in the art, that may be adapted for the treatment of PPHN as described herein.

In addition to the above general methods of making BH4, the present invention particularly contemplates making and using a BH4 composition which is a stabilized BH4 composition. Preferably the stabilized BH4 composition is in crystalline form. Methods of making the stabilized BH4 compositions for use in the present invention are described in Example 2. Such a crystalline form may prove useful as an additive to conventional infant formulas for the treatment of PPHN. The crystalline form also may conveniently be formed into a tablets, powder or other solid for oral administration. The forms and routes of administration of BH4 are discussed in further detail below.

In preferred embodiments, it is contemplated that the methods of the present invention will provide to a patient in need thereof, a daily dose of between about 10 mg/kg to about 20 mg/kg of BH4. Of course, one skilled in the art may adjust this dose up or down depending on the efficacy being achieved by the administration. The daily dose may be administered in a single dose or alternatively may be administered in multiple doses at conveniently spaced intervals. In exemplary embodiments, the daily dose may be 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 22 mg/kg, 24 mg/kg, 26 mg/kg, 28 mg/kg, 30 mg/kg, 32 mg/kg, 34 mg/kg, 36 mg/kg, 38 mg/kg, 40 mg/kg, 42 mg/kg, 44 mg/kg, 46 mg/kg, 48 mg/kg, 50 mg/kg, or more mg/kg.

Regardless of the amount of BH4 administered, it is desirable that the administration increases arterial oxygen pressures of the patients to the normal values discussed above.

### Combination Therapy

Certain methods of the invention involve the combined use of BH4 and conventional agents and interventions to effect a therapeutic outcome in patients with PPHN. To achieve the appropriate therapeutic outcome in the combination therapies contemplated herein, one would generally administer to the subject the BH4 composition and the agents/intervention in a combined amount effective to produce the desired therapeutic outcome (i.e., an increase in arterial oxygen pressures). This process may involve administering the BH4 composition and the agent/intervention at the same time. This may be achieved by administering a single composition or pharmacological formulation that includes both the therapeutic agent and BH4 or administering the BH4 formulation at the same time as the interventions is being conducted. Alternatively, the agent/intervention is taken at about the same time as a pharmacological formulation (tablet, injection or drink) of BH4. In other alternatives, the BH4 treatment may precede or follow the agent/intervention by intervals ranging from minutes to hours. In embodiments where the agent/intervention and the BH4 compositions are administered separately, one would generally ensure that both agents are exerting their effect concurrently, such that the BH4 will still be able to exert an advantageously effect on the patient. In such instances, it is contemplated that one would administer the BH4 within about 2-6 hours (before or after) of the agent/intervention, with a delay time of only about 1 hour being most preferred. However, it should be understood the 2-6 hour time frame between administration of the two agents is merely exemplary, it may be that longer time intervals, e.g., 24 hours, 36 hours, 48 hours, 72 hours, one week or more between administration of the BH4 and the second agent/intervention also is contemplated. In certain embodiments, it is contemplated that the BH4 therapy will be a continuous therapy where a daily dose of BH4 is administered to the patient indefinitely.

### Pharmaceutical Compositions

Pharmaceutical compositions for administration according to the present invention can comprise a first composition comprising BH4 in a pharmaceutically acceptable form optionally combined with a pharmaceutically acceptable carrier. These compositions can be administered by any means that achieve their intended purposes. Amounts and regimens for the administration of a composition according to the present invention can be determined readily by those with ordinary skill in the art for treating PPHN. As discussed above, those of skill in the art could initially employ amounts and regimens of BH4 currently being proposed in a medical context, e.g., those compositions that are being proposed for modulating NOS activity. Any of the protocols, formulations, routes of administration and the like described that have been used for administering BH4 for loading tests can readily be modified for use in the present invention.

Compositions within the scope of this invention include all compositions comprising BH4, analogs and derivative thereof according to the present invention in an amount effective to achieve its intended purpose. Similarly, as certain therapeutic methods of the present invention contemplate a combination therapy in which BH4-based compositions are administered in addition to agents and interventions commonly used to treat PPHN, the pharmaceutical compositions of the invention also contemplate all compositions comprising at least BH4-based therapeutic agent, analog or homologue thereof in an amount effective to achieve the amelioration of one or more of the symptoms of PPHN when administered in combination with the conventional agents and interventions used to treat PPHN. Of course, the most obvious symptom that may be alleviated is that the combined therapy produces an increase in arterial oxygen pressures, however, other symptoms such as hypoxemia, low Agar scores of 5 or less at 1 and 5 minutes, cyanosis, tachypnea, retractions, systolic murmur, mixed acidosis, hypercapnea, cardiomegaly, decreased pulmonary vasculature, large differences (greater than 15 mm Hg) between preductal and postductal arterial blood gases of infant on 100% O₂ that is subjected to hyperoxia test, and prolonged right ventricle PEP/ET ratio (greater than 50) as assessed by echocardiography and the like also may be monitored. Such indicia are monitored using techniques known to those of skill in the art.

### Crystal Polymorphs of (6R) L-Tetrahydrobiopterin Dihydrochloride Salt

It has been found that BH4, and in particular, the dihydrochloride salt of BH4, exhibits crystal polymorphism. The structure of BH4 is shown below:

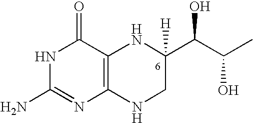

The (6R) form of BH4 is the known biologically active form, however, BH4 is also known to be unstable at ambient temperatures. It has been found that one crystal polymorph of BH4 is more stable, and is stable to decomposition under ambient conditions.

BH4 is difficult to handle and it is therefore produced and offered as its dihydrochloride salt (Schircks Laboratories, Jona, Switzerland) in ampoules sealed under nitrogen to prevent degradation of the substance due to its hygroscopic nature and sensitivity to oxidation. U.S. Pat. No. 4,649,197 discloses that separation of (6R)- and 6(S)-L-erythro-tetrahydrobiopterin dihydrochloride into its diastereomers is difficult due to the poor crystallinity of 6(R,S)-L-erythro-tetrahydrobiopterin dihydrochloride. The European patent number 0 079 574 describes the preparation of tetrahydrobiopterin, wherein a solid tetrahydrobiopterin dihydrochloride is obtained as an intermediate. S. Matsuura et al. describes in Chemistry Letters 1984, pages 735-738 and Heterocycles, Vol. 23, No. 12, 1985 pages 3115-3120 6(R)-tetrahydrobiopterin dihydrochloride as a crystalline solid in form of colorless needles, which are characterized by X-ray analysis disclosed in J. Biochem. 98, 1341-1348 (1985). An optical rotation of 6.81° was found the crystalline product, which is quite similar to the optical rotation of 6.51° reported for a crystalline solid in form of white crystals in example 6 of EP-A2-0 191 335.

Results obtained during development of (6R)-L-erythro-tetrahydrobiopterin dihydrochloride indicated that the compound may exist in different crystalline forms, including polymorphic forms and solvates. The continued interest in this area requires an efficient and reliable method for the preparation of the individual crystal forms of (6R)-L-erythro-tetrahydrobiopterin dihydrochloride and controlled crystallization conditions to provide crystal forms, that are preferably stable and easy to handle and to process in the manufacture and preparation of formulations, and that provide a high storage stability in substance form or as formulated product, or which provide less stable forms suitable as intermediates for controlled crystallization for the manufacture of stable forms.

### Polymorph Form B

The crystal polymorph that has been found to be the most stable is referred to herein as “form B,” or alternatively as “polymorph B.” Results obtained during investigation and development of (6R)-L-erythro-tetrahydrobiopterin dihydrochloride development revealed that there are several known crystalline solids have been prepared, but none have recognized the polymorphism and its effect on the stability of the BH4 crystals.

Polymorph B is a slightly hygroscopic anhydrate with the highest thermodynamic stability above about 20° C. Furthermore, form B can be easily processed and handled due to its thermal stability, possibility for preparation by targeted conditions, its suitable morphology and particle size. Melting point is near 260° C. (ΔHf>140 J/g), but no clear melting point can be detected due to decomposition prior and during melting. These outstanding properties renders polymorph form B especially feasible for pharmaceutical application, which are prepared at elevated temperatures. Polymorph B can be obtained as a fine powder with a particle size that may range from 0.2 μm to 500 μm.

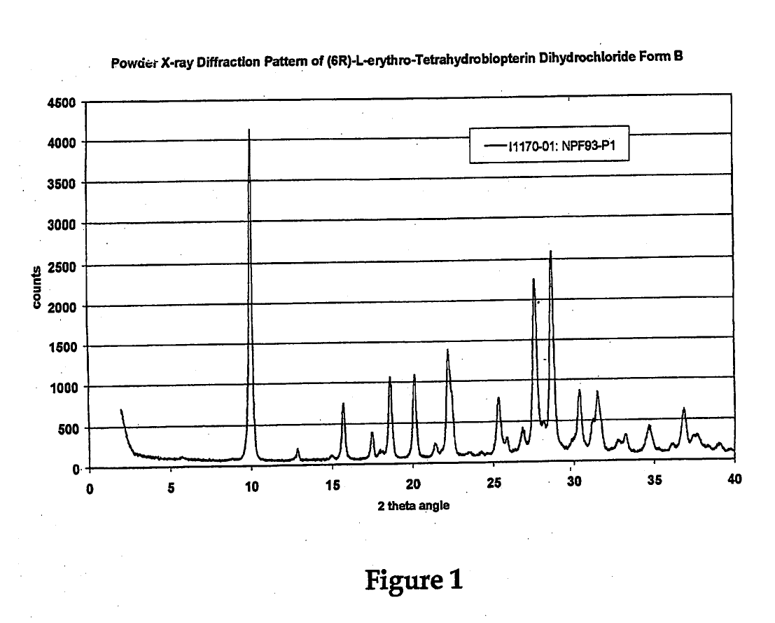

Form B exhibits an X-ray powder diffraction pattern, expressed in d-values (Å) at: 8.7 (vs), 6.9 (w), 5.90 (vw), 5.63 (m), 5.07 (m), 4.76 (m), 4.40 (m), 4.15 (w), 4.00 (s), 3.95 (m), 3.52 (m), 3.44 (w), 3.32 (m), 3.23 (s), 3.17 (w), 3.11 (vs), 3.06 (w), 2.99 (w), 2.96 (w), 2.94 (m), 2.87 (w), 2.84 (s), 2.82 (m), 2.69 (w), 2.59 (w), 2.44 (w). FIG. 1 is a graph of the characteristic X-ray diffraction pattern exhibited by form B of (6R)-L-erythro-tetrahydrobiopterin dihydrochloride.

As used herein, the following the abbreviations in brackets mean: (vs)=very strong intensity; (s)=strong intensity; (m)=medium intensity; (w)=weak intensity; and (vw)=very weak intensity. A characteristic X-ray powder diffraction pattern is exhibited in FIG. 1.

It has been found that other polymorphs of BH4 have a satisfactory chemical and physical stability for a safe handling during manufacture and formulation as well as providing a high storage stability in its pure form or in formulations. In addition, it has been found that form B, and other polymorphs of BH4 can be prepared in very large quantities (e.g., 100 kilo scale) and stored over an extended period of time.

All crystal forms (polymorphs, hydrates and solvates), inclusive crystal form B, can be used for the preparation of the most stable polymorph B. Polymorph B may be obtained by phase equilibration of suspensions of amorphous or other forms than polymorph form B, such as polymorph A, in suitable polar and non aqueous solvents. Thus, the pharmaceutical preparations described herein refers to a preparation of polymorph form B of (6R)-L-erythro-tetrahydrobiopterin dihydrochloride.

Other forms of BH4 can be converted for form B by dispersing the other form of BH4 in a solvent at room temperature, stirring the suspension at ambient temperatures for a time sufficient to produce polymorph form B, thereafter isolating crystalline form B and removing the solvent from the isolated form B. Ambient temperatures, as used herein, mean temperatures in a range from 0° C. to 60° C., preferably 15° C. to 40° C. The applied temperature may be changed during treatment and stirring by decreasing the temperature stepwise or continuously. Suitable solvents for the conversion of other forms to form B include but are not limited to, methanol, ethanol, isopropanol, other C3- and C4-alcohols, acetic acid, acetonitrile, tetrahydrofurane, methyl-t-butyl ether, 1,4-dioxane, ethyl acetate, isopropyl acetate, other C3-C6-acetates, methyl ethyl ketone and other methyl-C3-C5 alkyl-ketones. The time to complete phase equilibration may be up to 30 hours and preferably up to 20 hours or less than 20 hours.

Polymorph B may also be obtained by crystallisation from solvent mixtures containing up to about 5% water, especially from mixtures of ethanol, acetic acid and water. It has been found that polymorph form B of (6R)-L-erythro-tetrahydrobiopterin dihydrochloride can be prepared by dissolution, optionally at elevated temperatures, preferably of a solid lower energy form than form B or of form B of (6R)-L-erythro-tetrahydrobiopterin dihydrochloride in a solvent mixture comprising ethanol, acetic acid and water, addition of seeds to the solution, cooling the obtained suspension and isolation of the formed crystals. Dissolution may be carried out at room temperature or up to 70° C., preferably up to 50° C. There may be used the final solvent mixture for dissolution or the starting material may be first dissolved in water and the other solvents may than be added both or one after the other solvent. The composition of the solvent mixture may comprise a volume ratio of water:acetic acid:tetrahydrofuran of 1:3:2 to 1:9:4 and preferably 1:5:4. The solution is preferably stirred. Cooling may mean temperatures down to −40° C. to 0° C., preferably down to 10° C. to 30° C. Suitable seeds are polymorph form B from another batch or crystals having a similar or identical morphology. After isolation, the crystalline form B can be washed with a non-solvent such as acetone or tetrahydrofurane and dried in usual manner.

Polymorph B may also be obtained by crystallisation from aqueous solutions through the addition of non-solvents such as methanol, ethanol and acetic acid. The crystallisation and isolation procedure can be advantageously carried out at room temperature without cooling the solution. This process is therefore very suitable to be carried out at an industrial scale.

In one embodiment of the compositions and methods described herein, a composition including polymorph form B of (6R)-L-erythro-tetrahydrobiopterin dihydrochloride is prepared by dissolution of a solid form other than form B or of form B of (6R)-L-erythro-tetrahydrobiopterin dihydrochloride in water at ambient temperatures, adding a non-solvent in an amount sufficient to form a suspension, optionally stirring the suspension for a certain time, and thereafter isolation of the formed crystals. The composition is further modified into a pharmaceutical composition as described below.

The concentration of (6R)-L-erythro-tetrahydrobiopterin dihydrochloride in the aqueous solution may be from 10 to 80 percent by weight, more preferably from 20 to 60 percent by weight, by reference to the solution. Preferred non-solvents (i.e., solvents useful in preparing suspensions of BH4) are methanol, ethanol and acetic acid. The non-solvent may be added to the aqueous solution. More preferably, the aqueous solution is added to the non-solvent. The stirring time after formation of the suspension may be up to 30 hours and preferably up to 20 hours or less than 20 hours. Isolation by filtration and drying is carried out in known manner as described above.

Polymorph form B is a very stable crystalline form, that can be easily filtered off, dried and ground to particle sizes desired for pharmaceutical formulations. These outstanding properties render polymorph form B especially feasible for pharmaceutical application.

### Polymorph Form A

It has been found that another crystal polymorph of (6R)-L-erythro-tetrahydrobiopterin dihydrochloride is a stable preferred form of BH4 for use in a pharmaceutical preparation described herein, which shall be referred to herein as “form A,” or “polymorph A.” Polymorph A is slightly hygroscopic and adsorbs water to a content of about 3 percent by weight, which is continuously released between 50° C. and 200° C., when heated at a rate of 10° C./minute. The polymorph A is a hygroscopic anhydrate, which is a meta-stable form with respect to form B; however, it is stable over several months at ambient conditions if kept in a tightly sealed container. Form A is especially suitable as intermediate and starting material to produce stable polymorph forms. Polymorph form A can be prepared as a solid powder with desired medium particle size range which is typically ranging from 1 μm to about 500 μm.

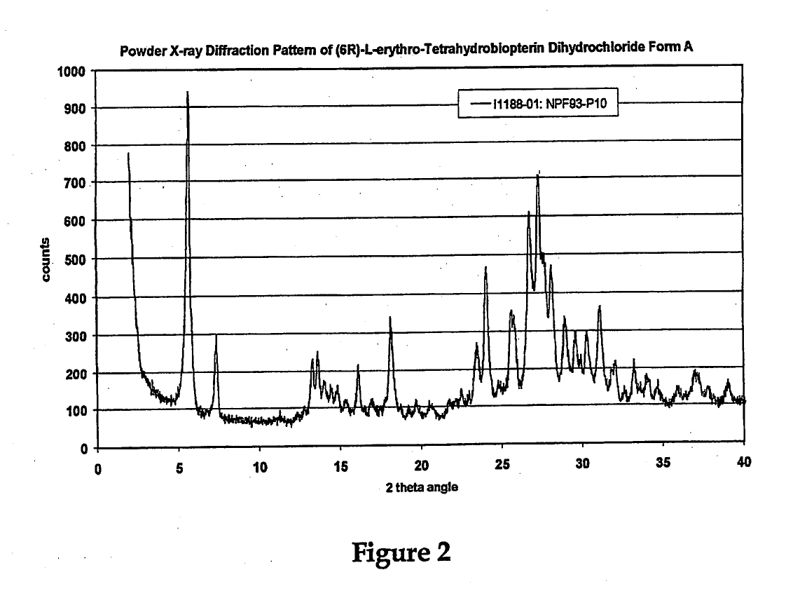

Polymorph A which exhibits a characteristic X-ray powder diffraction pattern with characteristic peaks expressed in d-values (Å) of: 15.5 (vs.), 12.0 (m), 6.7 (m), 6.5 (m), 6.3 (w), 6.1 (w), 5.96 (w), 5.49 (m), 4.89 (m), 3.79 (m), 3.70 (s), 3.48 (m), 3.45 (m), 3.33 (s), 3.26 (s), 3.22 (m), 3.18 (m), 3.08 (m), 3.02 (w), 2.95 (w), 2.87 (m), 2.79 (w), 2.70 (w). FIG. 2 is a graph of the characteristic X-ray diffraction pattern exhibited by form A of (6R)-L-erythro-tetrahydrobiopterin dihydrochloride.

Polymorph A exhibits a characteristic Raman spectra bands, expressed in wave numbers (cm-1) at: 2934 (w), 2880 (w), 1692 (s), 1683 (m), 1577 (w), 1462 (m), 1360 (w), 1237 (w), 1108 (w), 1005 (vw), 881 (vw), 813 (vw), 717 (m), 687 (m), 673 (m), 659 (m), 550 (w), 530 (w), 492 (m), 371 (m), 258 (w), 207 (w), 101 (s), 87 (s) cm-1.

Polymorph form A may be obtained by freeze-drying or water removal of solutions of (6R)-L-erythro-tetrahydrobiopterin dihydrochloride in water. Polymorph form A of (6R)-L-erythro-tetrahydrobiopterin dihydrochloride can be prepared by dissolving (6R)-L-erythro-tetrahydrobiopterin dihydrochloride at ambient temperatures in water, (1) cooling the solution to low temperatures for solidifying the solution, and removing water under reduced pressure, or (2) removing water from said aqueous solution.

The crystalline form A can be isolated by filtration and then dried to evaporate absorbed water from the product. Drying conditions and methods are known and drying of the isolated product or water removal pursuant to variant (2) described herein may be carried out in applying elevated temperatures, for example up to 80° C., preferably in the range from 30° C. to 80° C., under vacuum or elevated temperatures and vacuum. Prior to isolation of a precipitate obtained in variant (2), the suspension may be stirred for a certain time for phase equilibration. The concentration of (6R)-L-erythro-tetrahydrobiopterin dihydrochloride in the aqueous solution may be from 5 to 40 percent by weight, by reference to the solution.

A fast cooling is preferred to obtain solid solutions as starting material. A reduced pressure is applied until the solvent is completely removed. Freeze drying is a technology well known in the art. The time to complete solvent removal is dependent on the applied vacuum, which may be from 0.01 to 1 mbar, the solvent used and the freezing temperature.

Polymorph form A is stable at room temperature or below room temperature under substantially water free conditions, which is demonstrated with phase equilibration tests of suspensions in tetrahydrofuran or tertiary-butyl methyl ether stirred for five days and 18 hours respectively under nitrogen at room temperature. Filtration and air-drying at room temperature yields unchanged polymorph form A.

### Polymorph Form F

It has been found that another crystal polymorph of (6R)-L-erythro-tetrahydrobiopterin dihydrochloride is a stable preferred form of BH4 for use in a pharmaceutical preparation described herein, which shall be referred to herein as “form F,” or “polymorph F.” Polymorph F is slightly hygroscopic and adsorbs water to a content of about 3 percent by weight, which is continuously released between 50° C. and 200° C., when heated at a rate of 10° C./minute. The polymorph F is a meta-stable form and a hygroscopic anhydrate, which is more stable than form A at ambient lower temperatures and less stable than form B at higher temperatures and form F is especially suitable as intermediate and starting material to produce stable polymorph forms. Polymorph form F can be prepared as a solid powder with desired medium particle size range which is typically ranging from 1 μm to about 500 μm.

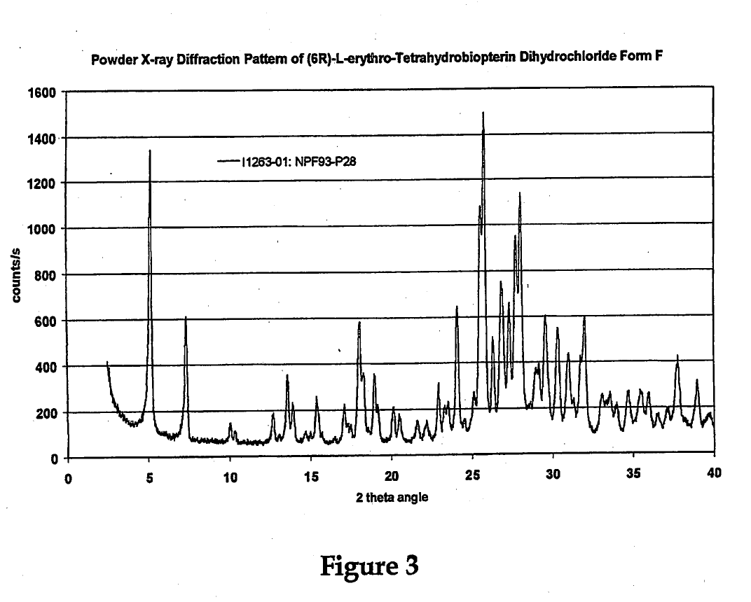

Polymorph F exhibits a characteristic X-ray powder diffraction pattern with characteristic peaks expressed in d-values (Å) at: 17.1 (vs.), 12.1 (w), 8.6 (w), 7.0 (w), 6.5 (w), 6.4 (w), 5.92 (w), 5.72 (w), 5.11 (w), 4.92 (m), 4.86 (w), 4.68 (m), 4.41 (w), 4.12 (w), 3.88 (w), 3.83 (w), 3.70 (m), 3.64 (w), 3.55 (m), 3.49 (s), 3.46 (vs), 3.39 (s), 3.33 (m), 3.31 (m), 3.27 (m), 3.21 (m), 3.19 (m), 3.09 (m), 3.02 (m), and 2.96 (m). FIG. 3 is a graph of the characteristic X-ray diffraction pattern exhibited by form F of (6R)-L-erythro-tetrahydrobiopterin dihydrochloride.

Polymorph F may be obtained by phase equilibration of suspensions of polymorph form A in suitable polar and non-aqueous solvents, which scarcely dissolve said lower energy forms, especially alcohols such as methanol, ethanol, propanol and isopropanol. Polymorph form F of (6R)-L-erythro-tetrahydrobiopterin dihydrochloride can also be prepared by dispersing particles of solid form A of (6R)-L-erythro-tetrahydrobiopterin dihydrochloride in a non-aqueous solvent that scarcely dissolves said (6R)-L-erythro-tetrahydrobiopterin dihydrochloride below room temperature, stirring the suspension at said temperatures for a time sufficient to produce polymorph form F, thereafter isolating crystalline form F and removing the solvent from the isolated form F. Removing of solvent and drying may be carried out under air, dry air or a dry protection gas such as nitrogen or noble gases and at or below room temperature, for example down to 0° C. The temperature during phase equilibration is preferably from 5 to 15° C. and most preferably about 10° C.

Polymorph Form J

It has been found that another crystal polymorph of (6R)-L-erythro-tetrahydrobiopterin dihydrochloride is a stable preferred form of BH4 for use in a pharmaceutical preparation described herein, which shall be referred to herein as “form J,” or “polymorph J.” The polymorph J is slightly hygroscopic and adsorbs water when handled at air humidity. The polymorph J is a meta-stable form and a hygroscopic anhydrate, and it can be transformed back into form E described below, from which it is obtained upon exposure to high relative humidity conditions such as above 75% relative humidity. Form J is especially suitable as intermediate and starting material to produce stable polymorph forms. Polymorph form J can be prepared as a solid powder with desired medium particle size range which is typically ranging from 1 μm to about 500 μm.

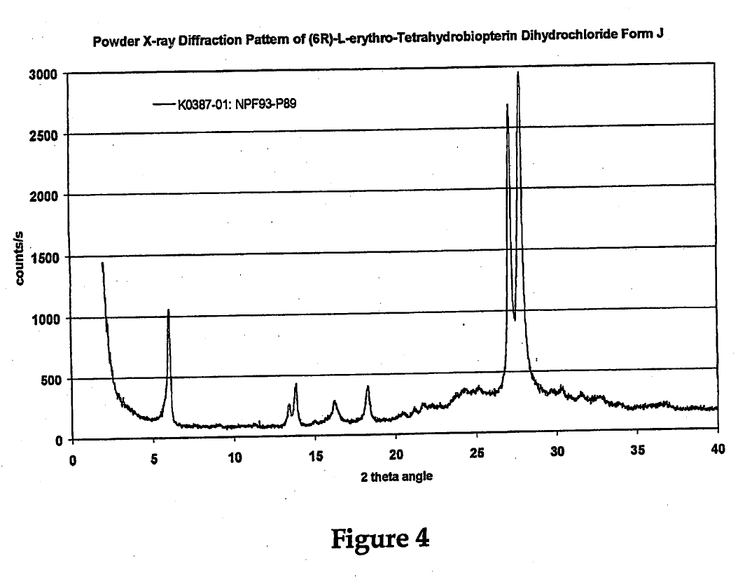

Form J exhibits a characteristic X-ray powder diffraction pattern with characteristic peaks expressed in d-values (Å) at: 14.6 (m), 6.6 (w), 6.4 (w), 5.47 (w), 4.84 (w), 3.29 (vs), and 3.21 (vs). FIG. 4 is a graph of the characteristic X-ray diffraction pattern exhibited by form J of (6R)-L-erythro-tetrahydrobiopterin dihydrochloride.

Polymorph J may be obtained by dehydration of form E at moderate temperatures under vacuum. In particular, polymorph form J of (6R)-L-erythro-tetrahydrobiopterin dihydrochloride can be prepared by taking form E and removing the water from form E by treating form E in a vacuum drier to obtain form J at moderate temperatures, which may mean a temperature in the range of 25 to 70° C., and most preferably 30 to 50° C.

### Polymorph Form K

It has been found that another crystal polymorph of (6R)-L-erythro-tetrahydrobiopterin dihydrochloride is a stable preferred form of BH4 for use in a pharmaceutical preparation described herein, which shall be referred to herein as “form K,” or “polymorph K.” Polymorph K is slightly hygroscopic and adsorbs water to a content of about 2.0 percent by weight, which is continuously released between 50° C. and 100° C., when heated at a rate of 10° C./minute. The polymorph K is a meta-stable form and a hygroscopic anhydrate, which is less stable than form B at higher temperatures and form K is especially suitable as intermediate and starting material to produce stable polymorph forms, in particular form B. Polymorph form K can be prepared as a solid powder with desired medium particle size range which is typically ranging from 1 μm to about 500 μm.

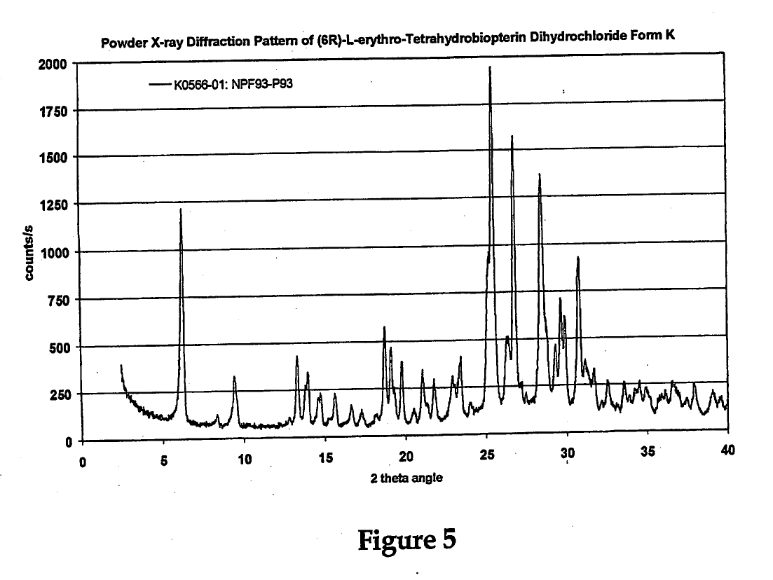

Form K exhibits a characteristic X-ray powder diffraction pattern with characteristic peaks expressed in d-values (Å) at: 14.0 (s), 9.4 (w), 6.6 (w), 6.4 (w), 6.3 (w), 6.1 (w), 6.0 (w), 5.66 (w), 5.33 (w), 5.13 (vw), 4.73 (m), 4.64 (m), 4.48 (w), 4.32 (vw), 4.22 (w), 4.08 (w), 3.88 (w), 3.79 (w), 3.54 (m), 3.49 (vs), 3.39 (m), 3.33 (vs), 3.13 (s), 3.10 (m), 3.05 (m), 3.01 (m), 2.99 (m), and 2.90 (m). FIG. 5 is a graph of the characteristic X-ray diffraction pattern exhibited by form K of (6R)-L-erythro-tetrahydrobiopterin dihydrochloride.

Polymorph K may be obtained by crystallization from mixtures of polar solvents containing small amounts of water and in the presence of small amounts of ascorbic acid. Solvents for the solvent mixture may be selected from acetic acid and an alcohol such as methanol, ethanol, n- or isopropanol. In particular, polymorph form K of (6R)-L-erythro-tetrahydrobiopterin dihydrochloride can be prepared by dissolving (6R)-L-erythro-tetrahydrobiopterin dihydrochloride in a mixture of acetic acid and an alcohol or tetrahydrofuran containing small amounts of water and a small amount of ascorbic acid at elevated temperatures, lowering temperature below room temperature to crystallize said dihydrochloride, isolating the precipitate and drying the isolated precipitate at elevated temperature optionally under vacuum. Suitable alcohols are for example methanol, ethanol, propanol and isopropanol, whereby ethanol is preferred. The ratio of acetic acid to alcohol or tetrahydrofuran may be from 2:1 to 1:2 and preferably about 1:1. Dissolution of (6R)-L-erythro-tetrahydrobiopterin dihydrochloride can be carried out in presence of a higher water content and more of the anti-solvent mixture can be added to obtain complete precipitation. The amount of water in the final composition may be from 0.5 to 5 percent by weight and the amount of ascorbic acid may be from 0.01 to 0.5 percent by weight, both by reference to the solvent mixture. The temperature for dissolution may be in the range from 30 to 100 and preferably 35 to 70° C. and the drying temperature may be in the range from 30 to 50° C. The precipitate may be washed with an alcohol such as ethanol after isolation, e.g., filtration. The polymorph K can easily be converted in the most stable form B by phase equilibration in e.g., isopropanol and optionally seeding with form B crystals at above room temperature such as temperatures from 30 to 40° C.

### Hydrate Forms of (6R) L-Tetrahydrobiopterin Dihydrochloride Salt

As further described below, it has been found that (6R)-L-erythro-tetrahydrobiopterin dihydrochloride exists as a number of crystalline hydrate, which shall be described and defined herein as forms C, D, E, H, and O. These hydrate forms are useful as a stable form of BH4 for the pharmaceutical preparations described herein and in the preparation of compositions including stable crystal polymorphs of BH4.

### Hydrate Form C

It has been found that a hydrate crystal form of (6R)-L-erythro-tetrahydrobiopterin dihydrochloride is a stable preferred form of BH4 for use in a pharmaceutical preparation described herein, which shall be referred to herein as “form C,” or “hydrate C.” The hydrate form C is slightly hygroscopic and has a water content of approximately 5.5 percent by weight, which indicates that form C is a monohydrate. The hydrate C has a melting point near 94° C. (ΔH_{f} is about 31 J/g) and hydrate form C is especially suitable as intermediate and starting material to produce stable polymorphic forms. Polymorph form C can be prepared as a solid powder with desired medium particle size range which is typically ranging from 1 μm to about 500 μm.

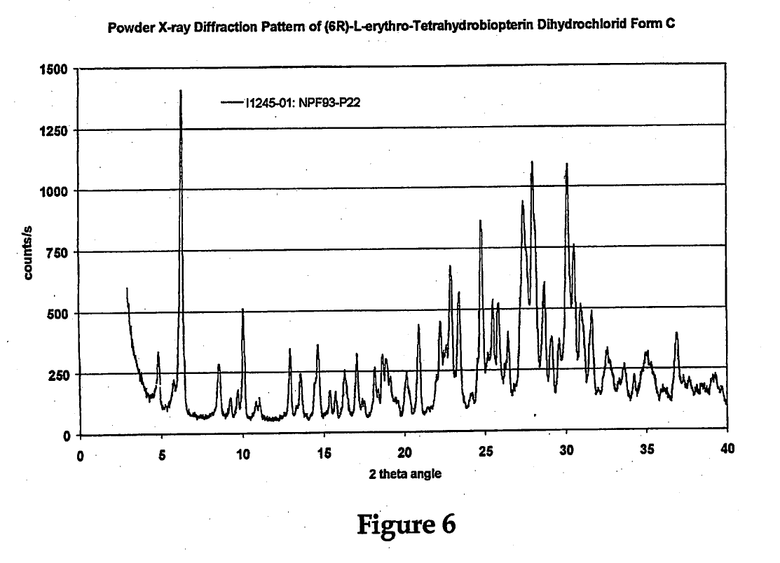

Form C exhibits a characteristic X-ray powder diffraction pattern with characteristic peaks expressed in d-values (Å) at: 18.2 (m), 15.4 (w), 13.9 (vs), 10.4 (w), 9.6 (w), 9.1 (w), 8.8 (m), 8.2 (w), 8.0 (w), 6.8 (m), 6.5 (w), 6.05 (m), 5.77 (w), 5.64 (w), 5.44 (w), 5.19 (w), 4.89 (w), 4.76 (w), 4.70 (w), 4.41 (w), 4.25 (m), 4.00 (m), 3.88 (m), 3.80 (m), 3.59 (s), 3.50 (m), 3.44 (m), 3.37 (m), 3.26 (s), 3.19 (vs), 3.17 (s), 3.11 (m), 3.06 (m), 3.02 (m), 2.97 (vs), 2.93 (m), 2.89 (m), 2.83 (m), and 2.43 (m). FIG. 6 is a graph of the characteristic X-ray diffraction pattern exhibited by hydrate form C of (6R)-L-erythro-tetrahydrobiopterin dihydrochloride.

Hydrate form C may be obtained by phase equilibration at ambient temperatures of a polymorph form such as polymorph B suspension in a non-solvent, which contains water in an amount of preferably about 5 percent by weight, by reference to the solvent. Hydrate form C of (6R)-L-erythro-tetrahydrobiopterin dihydrochloride cab be prepared by suspending (6R)-L-erythro-tetrahydrobiopterin dihydrochloride in a non-solvent such as, heptane, C1-C4-alcohols such as methanol, ethanol, 1- or 2-propanol, acetates, such as ethyl acetate, acetonitrile, acetic acid or ethers such as terahydrofuran, dioxane, tertiary-butyl methyl ether, or binary or ternary mixtures of such non-solvents, to which sufficient water is added to form a monohydrate, and stirring the suspension at or below ambient temperatures (e.g., 0 to 30° C.) for a time sufficient to form a monohydrate. Sufficient water may mean from 1 to 10 and preferably from 3 to 8 percent by weight of water, by reference to the amount of solvent. The solids may be filtered off and dried in air at about room temperature. The solid can absorb some water and therefore possess a higher water content than the theoretical value of 5.5 percent by weight. Hydrate form C is unstable with respect to forms D and B, and easily converted to polymorph form B at temperatures of about 40° C. in air and lower relative humidity. Form C can be transformed into the more stable hydrate D by suspension equilibration at room temperature.

### Hydrate Form D

It has been found that another hydrate crystal form of (6R)-L-erythro-tetrahydrobiopterin dihydrochloride is a stable preferred form of BH4 for use in a pharmaceutical preparation described herein, which shall be referred to herein as “form D,” or “hydrate D.” The hydrate form D is slightly hygroscopic and may have a water content of approximately 5.0 to 7.0 percent by weight, which suggests that form D is a monohydrate. The hydrate D has a melting point near 153° C. (ΔH_{f} is about 111 J/g) and is of much higher stability than form C and is even stable when exposed to air humidity at ambient temperature. Hydrate form D can therefore either be used to prepare formulations or as intermediate and starting material to produce stable polymorph forms. Polymorph form D can be prepared as a solid powder with desired medium particle size range which is typically ranging from 1 μm to about 500 μm.

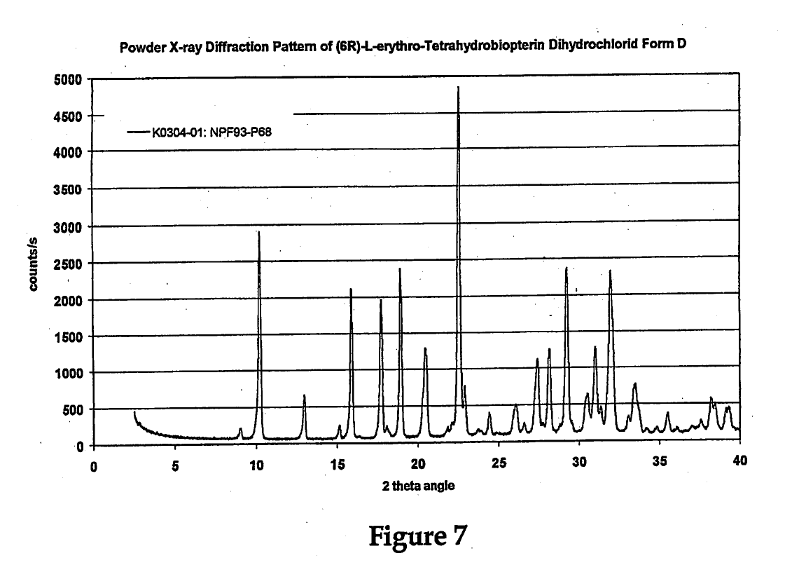

Form D exhibits a characteristic X-ray powder diffraction pattern with characteristic peaks expressed in d-values (Å) at: 8.6 (s), 6.8 (w), 5.56 (m), 4.99 (m), 4.67 (s), 4.32 (m), 3.93 (vs), 3.88 (w), 3.64 (w), 3.41 (w), 3.25 (w), 3.17 (m), 3.05 (s), 2.94 (w), 2.92 (w), 2.88 (m), 2.85 (w), 2.80 (w), 2.79 (m), 2.68 (w), 2.65 (w), 2.52 (vw), 2.35 (w), 2.34 (w), 2.30 (w), and 2.29 (w). FIG. 7 is a graph of the characteristic X-ray diffraction pattern exhibited by hydrate form D of (6R)-L-erythro-tetrahydrobiopterin dihydrochloride.

Hydrate form D may be obtained by adding at about room temperature concentrated aqueous solutions of (6R)-L-erythro-tetrahydrobiopterin dihydrochloride to an excess of a non-solvent such as hexane, heptane, dichloromethane, 1- or 2-propanol, acetone, ethyl acetate, acetonitrile, acetic acid or ethers such as terahydrofuran, dioxane, tertiary-butyl methyl ether, or mixtures of such non-solvents, and stirring the suspension at ambient temperatures. The crystalline solid can be filtered off and then dried under dry nitrogen at ambient temperatures. A preferred non-solvent is isopropanol. The addition of the aqueous solution may carried out drop-wise to avoid a sudden precipitation. Hydrate form D of (6R)-L-erythro-tetrahydrobiopterin dihydrochloride can be prepared by adding at about room temperature a concentrated aqueous solutions of (6R)-L-erythro-tetrahydrobiopterin dihydrochloride to an excess of a non-solvent and stirring the suspension at ambient temperatures. Excess of non-solvent may mean a ratio of aqueous to the non-solvent from 1:10 to 1:1000. Form D contains a small excess of water, related to the monohydrate, and it is believed that it is absorbed water due to the slightly hygroscopic nature of this crystalline hydrate. Hydrate form D is deemed to be the most stable one under the known hydrates at ambient temperatures and a relative humidity of less than 70%. Hydrate form D may be used for formulations prepared under conditions, where this hydrate is stable. Ambient temperature may mean 20 to 30° C.

### Hydrate Form E

It has been found that another hydrate crystal form of (6R)-L-erythro-tetrahydrobiopterin dihydrochloride is a stable preferred form of BH4 for use in a pharmaceutical preparation described herein, which shall be referred to herein as “form E,” or “hydrate E.” The hydrate form E has a water content of approximately 10 to 14 percent by weight, which suggests that form E is a dihydrate. The hydrate E is formed at temperatures below room temperature. Hydrate form E is especially suitable as intermediate and starting material to produce stable polymorph forms. It is especially suitable to produce the water-free form J upon drying under nitrogen or optionally under vacuum. Form E is non-hygroscopic and stable under rather high relative humidities, i.e., at relative humidities above about 60% and up to about 85%. Polymorph form E can be prepared as a solid powder with desired medium particle size range which is typically ranging from 1 μm to about 500 μm.

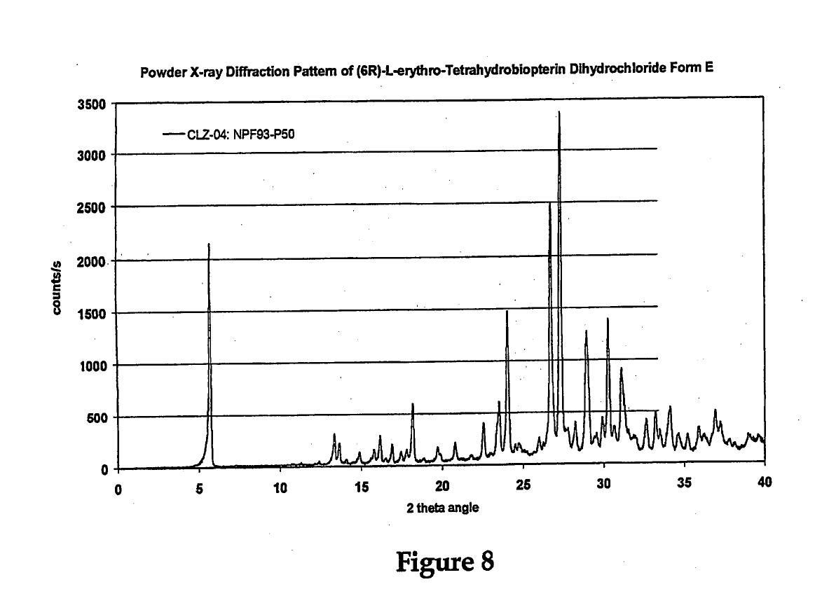

Form E exhibits a characteristic X-ray powder diffraction pattern with characteristic peaks expressed in d-values (Å) at: 15.4 (s), 6.6 (w), 6.5 (w), 5.95 (vw), 5.61 (vw), 5.48 (w), 5.24 (w), 4.87 (w), 4.50 (vw), 4.27 (w), 3.94 (w), 3.78 (w), 3.69 (m), 3.60 (w), 3.33 (s), 3.26 (vs), 3.16 (w), 3.08 (m), 2.98 (w), 2.95 (m), 2.91 (w), 2.87 (m), 2.79 (w), 2.74 (w), 2.69 (w), and 2.62 (w). FIG. 8 is a graph of the characteristic X-ray diffraction pattern exhibited by hydrate form E of (6R)-L-erythro-tetrahydrobiopterin dihydrochloride.

Hydrate form E may be obtained by adding concentrated aqueous solutions of (6R)-L-erythro-tetrahydrobiopterin dihydrochloride to an excess of a non-solvent cooled to temperatures from about 10 to −10° C. and preferably between 0 to 10° C. and stirring the suspension at said temperatures. The crystalline solid can be filtered off and then dried under dry nitrogen at ambient temperatures. Non-solvents are for example such as hexane, heptane, dichloromethane, 1- or 2-propanol, acetone, ethyl acetate, acetonitrile, acetic acid or ethers such as terahydrofuran, dioxane, tertiary-butyl methyl ether, or mixtures of such non-solvents. A preferred non-solvent is isopropanol. The addition of the aqueous solution may carried out drop-wise to avoid a sudden precipitation. Hydrate form E of (6R)-L-erythro-tetrahydrobiopterin dihydrochloride can be prepared by adding a concentrated aqueous solutions of (6R)-L-erythro-tetrahydrobiopterin dihydrochloride to an excess of a non-solvent, which is cooled to temperatures from about 10 to −10° C., and stirring the suspension at ambient temperatures. Excess of non-solvent may mean a ratio of aqueous to the non-solvent from 1:10 to 1:1000. A preferred non-solvent is tetrahydrofuran. Another preparation process comprises exposing polymorph form B to an air atmosphere with a relative humidity of 70 to 90%, preferably about 80%. Hydrate form E is deemed to be a dihydrate, whereby some additional water may be absorbed. Polymorph form E can be transformed into polymorph J upon drying under vacuum at moderate temperatures, which may mean between 20° C. and 50° C. at pressures between 0 and 100 mbar. Form E is especially suitable for formulations in semi solid forms because of its stability at high relative humidities.

### Hydrate Form H

It has been found that another hydrate crystal form of (6R)-L-erythro-tetrahydrobiopterin dihydrochloride is a stable preferred form of BH4 for use in a pharmaceutical preparation described herein, which shall be referred to herein as “form H,” or “hydrate H.” The hydrate form H has a water content of approximately 5.0 to 7.0 percent by weight, which suggests that form H is a hygroscopic monohydrate. The hydrate form H is formed at temperatures below room temperature. Hydrate form H is especially suitable as intermediate and starting material to produce stable polymorph forms. Polymorph form H can be prepared as a solid powder with desired medium particle size range which is typically ranging from 1 μm to about 500 μm.

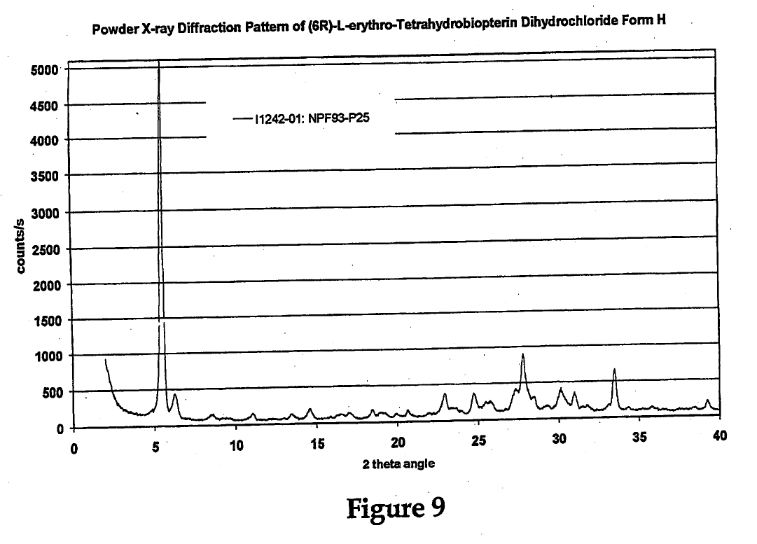

Form H exhibits a characteristic X-ray powder diffraction pattern with characteristic peaks expressed in d-values (Å) at: 8.6 15.8 (vs), 10.3 (w), 8.0 (w), 6.6 (w), 6.07 (w), 4.81 (w), 4.30 (w), 3.87 (m), 3.60 (m), 3.27 (m), 3.21 (m), 3.13 (w), 3.05 (w), 2.96 (m), 2.89 (m), 2.82 (w), and 2.67 (m). FIG. 9 is a graph of the characteristic X-ray diffraction pattern exhibited by hydrate form H of (6R)-L-erythro-tetrahydrobiopterin dihydrochloride.

Hydrate form H may be obtained by dissolving at ambient temperatures (6R)-L-erythro-tetrahydrobiopterin dihydrochloride in a mixture of acetic acid and water, adding then a non-solvent to precipitate a crystalline solid, cooling the obtained suspension and stirring the cooled suspension for a certain time. The crystalline solid is filtered off and then dried under vacuum at ambient temperatures. Non-solvents are for example such as hexane, heptane, dichloromethane, 1- or 2-propanol, acetone, ethyl acetate, acetonitril, acetic acid or ethers such as terahydrofuran, dioxane, tertiary-butyl methyl ether, or mixtures of such non-solvents. A preferred non-solvent is tetrahydrofuran. Hydrate form H of (6R)-L-erythro-tetrahydrobiopterin dihydrochloride can be by prepared by dissolving at ambient temperatures (6R)-L-erythro-tetrahydrobiopterin dihydrochloride in a mixture of acetic acid and a less amount than that of acetic acid of water, adding a non-solvent and cooling the obtained suspension to temperatures in the range of −10 to 10° C., and preferably −5 to 5° C., and stirring the suspension at said temperature for a certain time. Certain time may mean 1 to 20 hours. The weight ratio of acetic acid to water may be from 2:1 to 25:1 and preferably 5:1 to 15:1. The weight ratio of acetic acid/water to the non-solvent may be from 1:2 to 1:5. Hydrate form H seems to be a monohydrate with a slight excess of water absorbed due to the hygroscopic nature.

### Hydrate Form O

It has been found that another hydrate crystal form of (6R)-L-erythro-tetrahydrobiopterin dihydrochloride is a stable preferred form of BH4 for use in a pharmaceutical preparation described herein, which shall be referred to herein as “form O,” or “hydrate O.” The hydrate form O is formed at temperatures near room temperature. Hydrate form O is especially suitable as intermediate and starting material to produce stable polymorph forms. Polymorph form O can be prepared as a solid powder with desired medium particle size range which is typically ranging from 1 μm to about 500 μm.

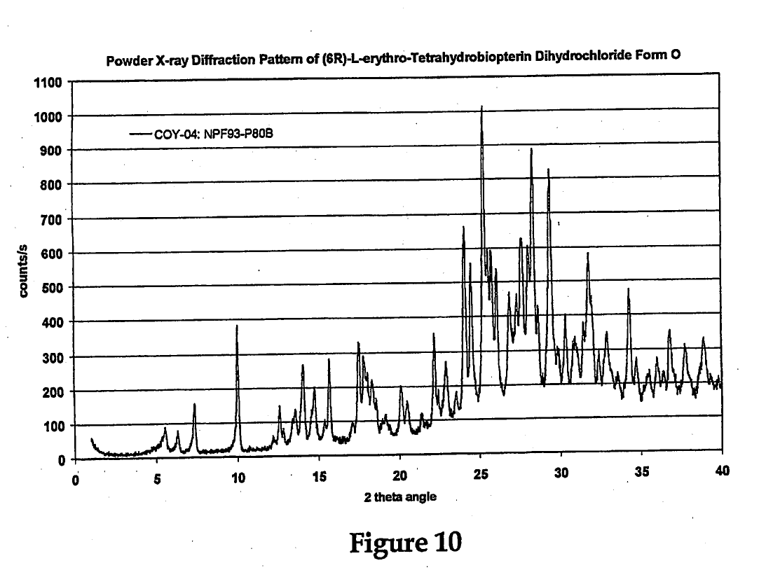

Form O exhibits a characteristic X-ray powder diffraction pattern with characteristic peaks expressed in d-values (Å) at: 15.9 (w), 14.0 (w), 12.0 (w), 8.8 (m), 7.0 (w), 6.5 (w), 6.3 (m), 6.00 (w), 5.75 (w), 5.65 (m), 5.06 (m), 4.98 (m), 4.92 (m), 4.84 (w), 4.77 (w), 4.42 (w), 4.33 (w), 4.00 (m), 3.88 (m), 3.78 (w), 3.69 (s), 3.64 (s), 3.52 (vs), 3.49 (s), 3.46 (s), 3.42 (s), 3.32 (m), 3.27 (m), 3.23 (s), 3.18 (s), 3.15 (vs), 3.12 (m), 3.04 (vs), 2.95 (m), 2.81 (s), 2.72 (m), 2.67 (m), and 2.61 (m). FIG. 10 is a graph of the characteristic X-ray diffraction pattern exhibited by hydrate form O of (6R)-L-erythro-tetrahydrobiopterin dihydrochloride.

Hydrate form O can be prepared by exposure of polymorphic form F to a nitrogen atmosphere containing water vapor with a resulting relative humidity of about 52% for about 24 hours. The fact that form F, which is a slightly hygroscopic anhydrate, can be used to prepare form O under 52% relative humidity suggests that form O is a hydrate, which is more stable than form F under ambient temperature and humidity conditions.

### Solvate Forms of (6R) L-Tetrahydrobiopterin Dihydrochloride Salt

As further described below, it has been found that (6R)-L-erythro-tetrahydrobiopterin dihydrochloride exists as a number of crystalline solvate forms, which shall be described and defined herein as forms G, I, L, M, and N. These solvate forms are useful as a stable form of BH4 for the pharmaceutical preparations described herein and in the preparation of compositions including stable crystal polymorphs of BH4.

### Solvate Form G

It has been found that an ethanol solvate crystal form of (6R)-L-erythro-tetrahydrobiopterin dihydrochloride is a stable preferred form of BH4 for use in a pharmaceutical preparation described herein, which shall be referred to herein as “form G,” or “hydrate G.” The ethanol solvate form G has a ethanol content of approximately 8.0 to 12.5 percent by weight, which suggests that form G is a hygroscopic mono ethanol solvate. The solvate form G is formed at temperatures below room temperature. Form G is especially suitable as intermediate and starting material to produce stable polymorph forms. Polymorph form G can be prepared as a solid powder with a desired medium particle size range which is typically ranging from 1 μm to about 500 μm.

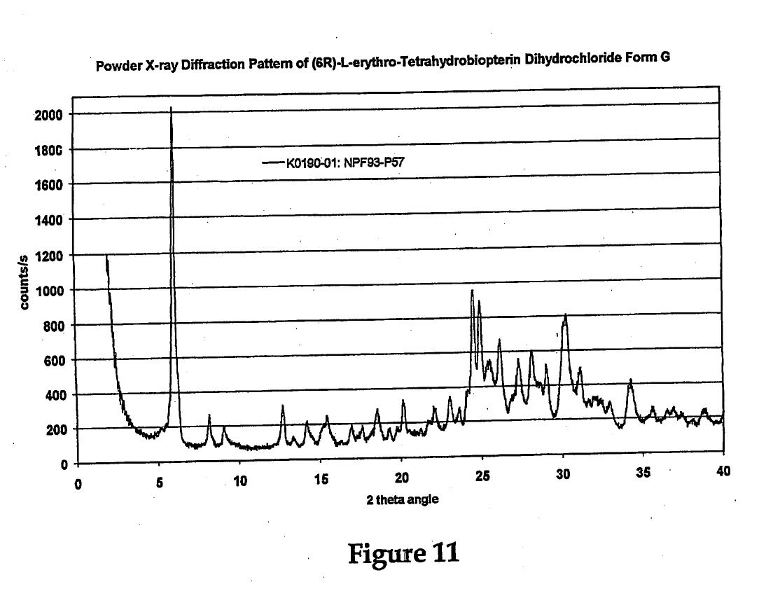

Form G exhibits a characteristic X-ray powder diffraction pattern with characteristic peaks expressed in d-values (Å) at: 14.5 (vs), 10.9 (w), 9.8 (w), 7.0 (w), 6.3 (w), 5.74 (w), 5.24 (vw), 5.04 (vw), 4.79 (w), 4.41 (w), 4.02 (w), 3.86 (w), 3.77 (w), 3.69 (w), 3.63 (m), 3.57 (m), 3.49 (m), 3.41 (m), 3.26 (m), 3.17 (m), 3.07 (m), 2.97 (m), 2.95 (m), 2.87 (w), and 2.61 (w). FIG. 11 is a graph of the characteristic X-ray diffraction pattern exhibited by solvate form G of (6R)-L-erythro-tetrahydrobiopterin dihydrochloride.

Ethanol solvate form G may be obtained by crystallization of L-erythro-tetrahydrobiopterin dihydrochloride dissolved in water and adding a large excess of ethanol, stirring the obtained suspension at or below ambient temperatures and drying the isolated solid under air or nitrogen at about room temperature. Here, a large excess of ethanol means a resulting mixture of ethanol and water with less than 10% water, preferably about 3 to 6%. Ethanolate form G of (6R)-L-erythro-tetrahydrobiopterin dihydrochloride can be prepared by dissolving at about room temperature to temperatures of 75° C. (6R)-L-erythro-tetrahydrobiopterin dihydrochloride in water or in a mixture of water and ethanol, cooling a heated solution to room temperature and down to 5 to 10° C., adding optionally ethanol to complete precipitation, stirring the obtained suspension at temperatures of 20 to 5° C., filtering off the white, crystalline solid and drying the solid under air or a protection gas such as nitrogen at temperatures about room temperature. The process may be carried out in a first variant in dissolving (6R)-L-erythro-tetrahydrobiopterin dihydrochloride at about room temperature in a lower amount of water and then adding an excess of ethanol and then stirring the obtained suspension for a time sufficient for phase equilibration. In a second variant, (6R)-L-erythro-tetrahydrobiopterin dihydrochloride may be suspended in ethanol, optionally adding a lower amount of water, and heating the suspension and dissolute (6R)-L-erythro-tetrahydrobiopterin dihydrochloride, cooling down the solution to temperatures of about 5 to 15° C., adding additional ethanol to the suspension and then stirring the obtained suspension for a time sufficient for phase equilibration.

### Solvate Form I

It has been found that an acetic acid solvate crystal form of (6R)-L-erythro-tetrahydrobiopterin dihydrochloride is a stable preferred form of BH4 for use in a pharmaceutical preparation described herein, which shall be referred to herein as “form I,” or “hydrate I.” The acetic acid solvate form I has an acetic acid content of approximately 12.7 percent by weight, which suggests that form I is a hygroscopic acetic acid mono solvate. The solvate form I is formed at temperatures below room temperature. Acetic acid solvate form I is especially suitable as intermediate and starting material to produce stable polymorph forms. Polymorph form I can be prepared as a solid powder with desired medium particle size range which is typically ranging from 1 μm to about 500 μm.

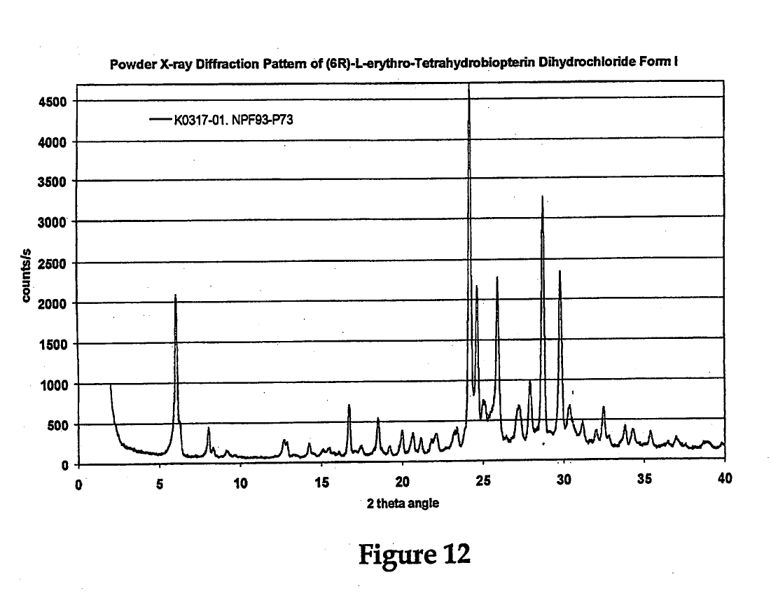

Form I exhibits a characteristic X-ray powder diffraction pattern with characteristic peaks expressed in d-values (Å) at: 14.5 (m), 14.0 (w), 11.0 (w), 7.0 (vw), 6.9 (vw), 6.2 (vw), 5.30 (w), 4.79 (w), 4.44 (w), 4.29 (w), 4.20 (vw), 4.02 (w), 3.84 (w), 3.80 (w), 3.67 (vs), 3.61 (m), 3.56 (w), 3.44 (m), 3.27 (w), 3.19 (w), 3.11 (s), 3.00 (m), 2.94 (w), 2.87 (w), and 2.80 (w). FIG. 12 is a graph of the characteristic X-ray diffraction pattern exhibited by solvate form I of (6R)-L-erythro-tetrahydrobiopterin dihydrochloride.

Acetic acid solvate form I may be obtained by dissolution of L-erythro-tetrahydrobiopterin dihydrochloride in a mixture of acetic acid and water at elevated temperature, adding further acetic acid to the solution, cooling down to a temperature of about 10° C., then warming up the formed suspension to about 15° C., and then stirring the obtained suspension for a time sufficient for phase equilibration, which may last up to 3 days. The crystalline solid is then filtered off and dried under air or a protection gas such as nitrogen at temperatures about room temperature.

### Solvate Form L

It has been found that a mixed ethanol solvate/hydrate crystal form of (6R)-L-erythro-tetrahydrobiopterin dihydrochloride is a stable preferred form of BH4 for use in a pharmaceutical preparation described herein, which shall be referred to herein as “form L,” or “hydrate L.” Form L may contain 4% but up to 13% ethanol and 0% to about 6% of water. Form L may be transformed into form G when treated in ethanol at temperatures from about 0° C. to 20° C. In addition form L may be transformed into form B when treated in an organic solvent at ambient temperatures (10° C. to 60° C.). Polymorph form L can be prepared as a solid powder with desired medium particle size range which is typically ranging from 1 μm to about 500 μm.

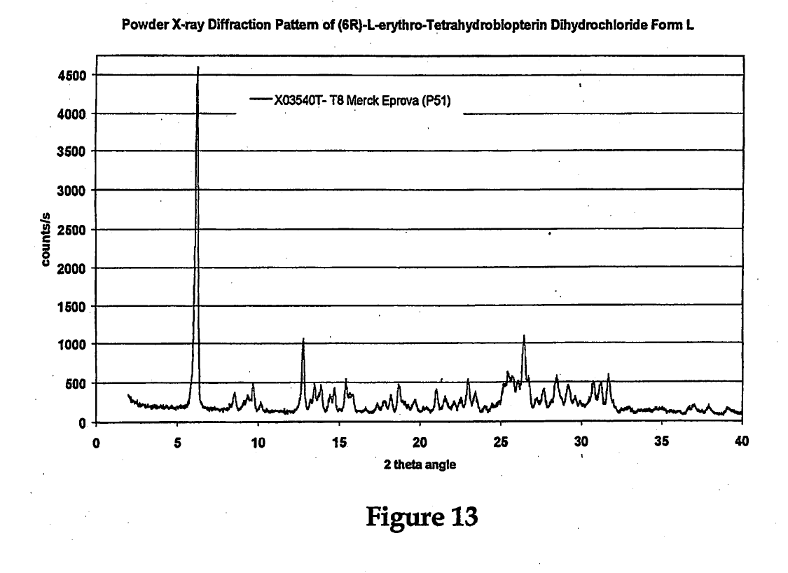

Form L exhibits a characteristic X-ray powder diffraction pattern with characteristic peaks expressed in d-values (Å) at: 14.1 (vs), 10.4 (w), 9.5 (w), 9.0 (vw), 6.9 (w), 6.5 (w), 6.1 (w), 5.75 (w), 5.61 (w), 5.08 (w), 4.71 (w), 3.86 (w), 3.78 (w), 3.46 (m), 3.36 (m), 3.06 (w), 2.90 (w), and 2.82 (w). FIG. 13 is a graph of the characteristic X-ray diffraction pattern exhibited by solvate form L of (6R)-L-erythro-tetrahydrobiopterin dihydrochloride.

Form L may be obtained by suspending hydrate form E at room temperature in ethanol and stirring the suspension at temperatures from 0 to 10° C., preferably about 5° C., for a time sufficient for phase equilibration, which may be 10 to 20 hours. The crystalline solid is then filtered off and dried preferably under reduced pressure at 30° C. or under nitrogen. Analysis by TG-FTIR suggests that form L may contain variable amounts of ethanol and water, i.e., it can exist as an polymorph (anhydrate), as a mixed ethanol solvate/hydrate, or even as a hydrate.

### Solvate Form M

It has been found that an ethanol solvate crystal form of (6R)-L-erythro-tetrahydrobiopterin dihydrochloride is a stable preferred form of BH4 for use in a pharmaceutical preparation described herein, which shall be referred to herein as “form M,” or “hydrate M.” Form M may contain 4% but up to 13% ethanol and 0% to about 6% of water, which suggests that form M is a slightly hygroscopic ethanol solvate. The solvate form M is formed at room temperature. Form M is especially suitable as intermediate and starting material to produce stable polymorph forms, since form M can be transformed into form G when treated in ethanol at temperatures between about −10° to 15° C., and into form B when treated in organic solvents such as ethanol, C3 and C4 alcohols, or cyclic ethers such as THF and dioxane. Polymorph form M can be prepared as a solid powder with desired medium particle size range which is typically ranging from 1 μm to about 500 μm.

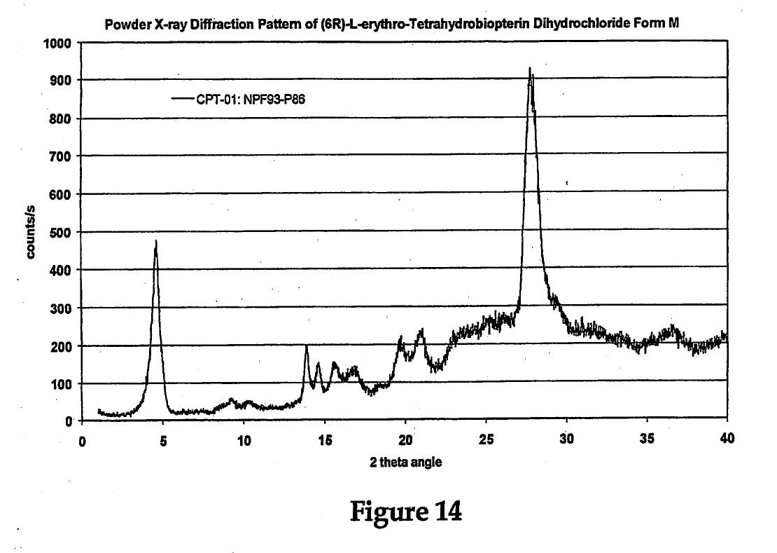

Form M exhibits a characteristic X-ray powder diffraction pattern with characteristic peaks expressed in d-values (Å) at: 18.9 (s), 6.4 (m), 6.06 (w), 5.66 (w), 5.28 (w), 4.50 (w), 4.23 (w), and 3.22 (vs). FIG. 14 is a graph of the characteristic X-ray diffraction pattern exhibited by solvate form M of (6R)-L-erythro-tetrahydrobiopterin dihydrochloride.

Ethanol solvate form M may be obtained by dissolution of L-erythro-tetrahydrobiopterin dihydrochloride in ethanol and evaporation of the solution under nitrogen at ambient temperature, i.e., between 10° C. and 40° C. Form M may also be obtained by drying of form G under a slight flow of dry nitrogen at a rate of about 20 to 100 ml/min. Depending on the extent of drying under nitrogen, the remaining amount of ethanol may be variable, i.e., from about 3% to 13%.

### Solvate Form N

It has been found that another solvate crystal form of (6R)-L-erythro-tetrahydrobiopterin dihydrochloride is a stable preferred form of BH4 for use in a pharmaceutical preparation described herein, which shall be referred to herein as “form N,” or “hydrate N.” Form N may contain in total up to 10% of isopropanol and water, which suggests that form N is a slightly hygroscopic isopropanol solvate. Form N may be obtained through washing of form D with isopropanol and subsequent drying in vacuum at about 30° C. Form N is especially suitable as intermediate and starting material to produce stable polymorph forms. Polymorph form N can be prepared as a solid powder with desired medium particle size range which is typically ranging from 1 μm to about 500 μm.

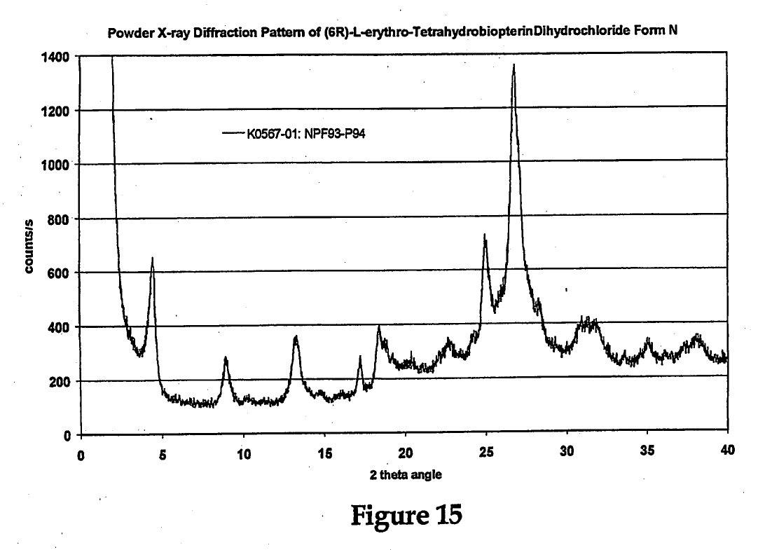

Form N exhibits a characteristic X-ray powder diffraction pattern with characteristic peaks expressed in d-values (Å) at: 19.5 (m), 9.9 (w), 6.7 (w), 5.15 (w), 4.83 (w), 3.91 (w), 3.56 (m), 3.33 (vs), 3.15 (w), 2.89 (w), 2.81 (w), 2.56 (w), and 2.36 (w). FIG. 15 is a graph of the characteristic X-ray diffraction pattern exhibited by solvate form N of (6R)-L-erythro-tetrahydrobiopterin dihydrochloride.

The isopropanol form N may be obtained by dissolution of L-erythro-tetrahydrobiopterin dihydrochloride in 4.0 ml of a mixture of isopropanol and water (mixing volume ratio for example 4:1). To this solution is slowly added isopropanol (IPA, for example about 4.0 ml) and the resulting suspension is cooled to 0° C. and stirred for several hours (e.g., about 10 to 18 hours) at this temperature. The suspension is filtered and the solid residue washed with isopropanol at room temperature. The obtained crystalline material is then dried at ambient temperature (e.g., about 20 to 30° C.) and reduced pressure (about 2 to 10 mbar) for several hours (e.g., about 5 to 20 hours). TG-FTIR shows a weight loss of 9.0% between 25 to 200° C., which is attributed to both isopropanol and water. This result suggests that form N can exist either in form of an isopropanol solvate, or in form of mixed isopropanol solvate/hydrate, or as an non-solvated form containing a small amount of water.

For the preparation of the polymorph forms, there may be used crystallization techniques well known in the art, such as stirring of a suspension (phase equilibration in), precipitation, re-crystallization, evaporation, solvent like water sorption methods or decomposition of solvates. Diluted, saturated or super-saturated solutions may be used for crystallization, with or without seeding with suitable nucleating agents. Temperatures up to 100° C. may be applied to form solutions. Cooling to initiate crystallization and precipitation down to −100° C. and preferably down to −30° C. may be applied. Meta-stable polymorphs or pseudo-polymorphic forms can be used to prepare solutions or suspensions for the preparation of more stable forms and to achieve higher concentrations in the solutions.

It was surprisingly found that hydrate form D is the most stable form under the hydrates and forms B and D are especially suitable to be used in pharmaceutical formulations. Forms B and D presents some advantages like an aimed manufacture, good handling due to convenient crystal size and morphology, very good stability under production conditions of various types of formulation, storage stability, higher solubility, and high bioavailability. Accordingly, one embodiment of the compositions and methods disclosed herein is pharmaceutical composition including polymorph form B and/or hydrate form D of (6R)-L-erythro-tetrahydrobiopterin dihydrochloride and a pharmaceutically acceptable carrier or diluent.

The crystal forms of (6R)-L-erythro-tetrahydrobiopterin dihydrochloride may be used together with folic acid or tetrahydrofolic acid or their pharmaceutically acceptable salts such as sodium, potassium, calcium or ammonium salts, each alone or additionally with arginine. The weight ratio of crystal forms: folic acids or salts thereof: arginine may be from about 1:10:10 to about 10:1:1.

The invention provides methods of using any of the tetrahydrobiopterin polymorphs described herein, or stable pharmaceutical preparations comprising any of such polymorphs, for treatment of conditions associated with reduced arterial oxygen pressures, most particularly PPHN. Concurrent treatment with folates, including folate precursors, folic acids, or folate derivatives, is also contemplated, as is treatment with a pharmaceutical composition or foodstuff that comprises both a tetrahydrobiopterin polymorph and a folate. Exemplary folates are disclosed in U.S. Pat. Nos. 6,011,040 and 6,544,994, both of which are incorporated herein by reference, and include folic acid (pteroylmonoglutamate), dihydrofolic acid, tetrahydrofolic acid, 5-methyltetrahydrofolic acid, 5,10-methylenetetrahydrofolic acid, 5,10-methenyltetrahydrofolic acid, 5,10-formiminotetahydrofolic acid, 5-formyltetrahydrofolic acid (leucovorin), 10-formyltetrahydrofolic acid, 10-methyltetrahydrofolic acid, one or more of the folylpolyglutamates, compounds in which the pyrazine ring of the pterin moiety of folic acid or of the folylpolyglutamates is reduced to give dihydrofolates or tetrahydrofolates, or derivatives of all the preceding compounds in which the N-5 or N-10 positions carry one carbon units at various levels of oxidation, or pharmaceutically compatible salts thereof, or a combination of two or more thereof. Exemplary tetrahydrofolates include 5-formyl-(6S)-tetrahydrofolic acid, 5-methyl-(6S)-tetrahydrofolic acid, 5,10-methylene-(6R)-tetrahydrofolic acid, 5,10-methenyl-(6R)-tetrahydrofolic acid, 10-formyl-(6R)-tetrahydrofolic acid, 5-formimino-(6S)-tetrahydrofolic acid or (6S)-tetrahydrofolic acid, and salts thereof.

### Pharmaceutical Formulations

The formulations described herein are preferably administered as oral formulations. Oral formulations are preferably solid formulations such as capsules, tablets, pills and troches, or liquid formulations such as aqueous suspensions, elixirs and syrups. The various form of BH4 described herein can be directly used as powder (micronized particles), granules, suspensions or solutions, or it may be combined together with other pharmaceutically acceptable ingredients in admixing the components and optionally finely divide them, and then filling capsules, composed for example from hard or soft gelatin, compressing tablets, pills or troches, or suspend or dissolve them in carriers for suspensions, elixirs and syrups. Coatings may be applied after compression to form pills.

Pharmaceutically acceptable ingredients are well known for the various types of formulation and may be for example binders such as natural or synthetic polymers, excipients, lubricants, surfactants, sweetening and flavoring agents, coating materials, preservatives, dyes, thickeners, adjuvants, antimicrobial agents, antioxidants and carriers for the various formulation types. Nonlimiting examples of binders useful in a composition described herein include gum tragacanth, acacia, starch, gelatin, and biological degradable polymers such as homo- or co-polyesters of dicarboxylic acids, alkylene glycols, polyalkylene glycols and/or aliphatic hydroxyl carboxylic acids; homo- or co-polyamides of dicarboxylic acids, alkylene diamines, and/or aliphatic amino carboxylic acids; corresponding polyester-polyamide-co-polymers, polyanhydrides, polyorthoesters, polyphosphazene and polycarbonates. The biological degradable polymers may be linear, branched or crosslinked. Specific examples are poly-glycolic acid, poly-lactic acid, and poly-d,l-lactide/glycolide. Other examples for polymers are water-soluble polymers such as polyoxaalkylenes (polyoxaethylene, polyoxapropylene and mixed polymers thereof, poly-acrylamides and hydroxylalkylated polyacrylamides, poly-maleic acid and esters or -amides thereof, poly-acrylic acid and esters or -amides thereof, poly-vinylalcohol und esters or -ethers thereof, poly-vinylimidazole, poly-vinylpyrrolidon, und natural polymers like chitosan.

Nonlimiting examples of excipients useful in a composition described herein include phosphates such as dicalcium phosphate. Nonlimiting examples of lubricants use in a composition described herein include natural or synthetic oils, fats, waxes, or fatty acid salts such as magnesium stearate.

Surfactants for use in a composition described herein can be anionic, anionic, amphoteric or neutral. Nonlimiting examples of surfactants useful in a composition described herein include lecithin, phospholipids, octyl sulfate, decyl sulfate, dodecyl sulfate, tetradecyl sulfate, hexadecyl sulfate and octadecyl sulfate, Na oleate or Na caprate, 1-acylaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid, and 1-octadecanoylaminoethane-2-sulfonic acid, and taurocholic acid and taurodeoxycholic acid, bile acids and their salts, such as cholic acid, deoxycholic acid and sodium glycocholates, sodium caprate or sodium laurate, sodium oleate, sodium lauryl sulphate, sodium cetyl sulphate, sulfated castor oil and sodium dioctylsulfosuccinate, cocamidopropylbetaine and laurylbetaine, fatty alcohols, cholesterols, glycerol mono- or -distearate, glycerol mono- or -dioleate and glycerol mono- or -dipalmitate, and polyoxyethylene stearate.

Nonlimiting examples of sweetening agents useful in a composition described herein include sucrose, fructose, lactose or aspartame. Nonlimiting examples of flavoring agents for use in a composition described herein include peppermint, oil of wintergreen or fruit flavors such as cherry or orange flavor. Nonlimiting examples of coating materials for use in a composition described herein include gelatin, wax, shellac, sugar or other biological degradable polymers. Nonlimiting examples of preservatives for use in a composition described herein include methyl or propylparabens, sorbic acid, chlorobutanol, phenol and thimerosal.

The hydrate form D described herein may also be formulated as effervescent tablet or powder, which disintegrate in an aqueous environment to provide a drinking solution. A syrup or elixir may contain the polymorph described herein, sucrose or fructose as sweetening agent a preservative like methylparaben, a dye and a flavoring agent.

Slow release formulations may also be prepared from the polymorph described herein in order to achieve a controlled release of the active agent in contact with the body fluids in the gastro intestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

While individual needs vary, determination of optimal ranges of effective amounts of each component is within the skill of the art. Typical dosages of the BH4 comprise about 1 to about 20 mg/kg body weight per day, which will usually amount to about 5 (1 mg/kg×5 kg body weight) to 3000 mg/day (30 mg/kg×100 kg body weight). Such a dose may be administered in a single dose or it may be divided into multiple doses. While continuous, daily administration is contemplated, it may be desirable to ceases the BH4 therapy when arterial oxygen pressures are improved to above a certain threshold level. Of course, the therapy may be reinitiated in the event that arterial oxygen pressures fall again.

It is understood that the suitable dose of a composition according to the present invention will depend upon the age, health and weight of the recipient, kind of concurrent treatment, if any, frequency of treatment, and the nature of the effect desired (i.e., the amount of decrease in pulmonary pressures desired). The frequency of dosing also is dependent on pharmacodynamic effects on arterial oxygen pressures. However, the most preferred dosage can be tailored to the individual subject, as is understood and determinable by one of skill in the art, without undue experimentation. This typically involves adjustment of a standard dose, e.g., reduction of the dose if the patient has a low body weight.

As discussed above, the total dose required for each treatment may be administered in multiple doses or in a single dose. The BH4 compositions may be administered alone or in conjunction with other therapeutics directed to the disease or directed to other symptoms thereof.

As is apparent from the disclosure presented herein, in a broad aspect the present application contemplates clinical application of a composition that contains a crystallized BH4 formulation. The compositions should be formulated into suitable pharmaceutical compositions, i.e., in a form appropriate for in vivo applications in such combination therapies. Generally, this will entail preparing compositions that are essentially free of pyrogens, as well as other impurities that could be harmful to humans or animals. Preferably, the formulation comprising the crystallized BH4 composition may be such that it can be used directly for the treatment of PPHN.

One will generally desire to employ appropriate salts and buffers to render the BH4 suitable for uptake. Aqueous compositions of the present invention comprise an effective amount of the BH4 dissolved or dispersed in a pharmaceutically acceptable carrier or aqueous medium. Such compositions may be administered orally or via injection.

The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the therapeutic compositions, its use in therapeutic compositions is contemplated. Supplementary active ingredients also can be incorporated into the compositions. In exemplary embodiments, the medical protein formulation may comprise corn syrup solids, high-oleic safflower oil, coconut oil, soy oil, L-leucine, calcium phosphate tribasic, L-tyrosine, L-proline, L-lysine acetate, DATEM (an emulsifier), L-glutamine, L-valine, potassium phosphate dibasic, L-isoleucine, L-arginine, L-alanine, glycine, L-asparagine monohydrate, L-serine, potassium citrate, L-threonine, sodium citrate, magnesium chloride, L-histidine, L-methionine, ascorbic acid, calcium carbonate, L-glutamic acid, L-cystine dihydrochloride, L-tryptophan, L-aspartic acid, choline chloride, taurine, m-inositol, ferrous sulfate, ascorbyl palmitate, zinc sulfate, L-carnitine, alpha-tocopheryl acetate, sodium chloride, niacinamide, mixed tocopherols, calcium pantothenate, cupric sulfate, thiamine chloride hydrochloride, vitamin A palmitate, manganese sulfate, riboflavin, pyridoxine hydrochloride, folic acid, beta-carotene, potassium iodide, phylloquinone, biotin, sodium selenate, chromium chloride, sodium molybdate, vitamin D3 and cyanocobalamin. The amino acids, minerals and vitamins in the supplement should be provided in amounts that provide the recommended daily doses of each of the components.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

The active compositions of the present invention include classic pharmaceutical preparations of BH4, which have been discussed herein as well as those known to those of skill in the art. Administration of these compositions according to the present invention will be via any common route for dietary supplementation. The protein is preferably administered orally, as is the BH4.

In certain embodiments, it is contemplated that BH4 or precursors or derivatives thereof used for the treatment of PPHN are formulated as an inhalable formulation for administration through inhalation. As such, the BH4 or precursors or derivatives thereof may be prepared as an aerosol formulation. Methods to the treatment of pulmonary hypertension using inhalable compositions are known to those of skill in the art and are described, for example, in U.S. Pat. No. 6,756,033 (incorporated herein by reference), which provides a teaching of treatment of pulmonary hypertension by delivering prostaglandin preparations by inhalation. The inhalation techniques described in the aforementioned patent for prostaglandins also will be useful in producing inhalable preparations of BH4 and/or its precursors and derivatives. In addition, it is contemplated that PPHN may be treated by a combined administration of BH4-based compositions and prostaglandin preparations.

The active compounds may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The BH4 compositions may be prepared as pharmaceutical forms suitable for injectable use. Such compositions include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

A preferred formulation for the compositions of BH4 and for use with the methods described herein is a tablet formulation. It has surprisingly been found that the addition of ascorbic acid to a tablet formulation increase the stability of the formulation. Without intending to be limited to a particular mechanism of stabilization, it is believed that when the BH4 is mixed into a pharmaceutical formulation with a variety of excipients that the even a small amount of ascorbic acid (e.g., less than 2% by weight) creates a complex with the BH4 and inhibits one or more pathways in which the BH4 is degraded. Thus, as set forth in greater detail in Example 4, a preferred tablet formulation of BH4 for use herein includes ascorbic acid.

The BH4 used in a composition described herein is preferably formulated as a dihydrochloride salt, however, it is contemplated that other salt forms of BH4 posses the desired biological activity, and consequently, other salt forms of BH4 can be used.

Pharmaceutically acceptable base addition salts may be formed with metals or amines, such as alkali and alkaline earth metals or organic amines Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible. Examples of metals used as cations are sodium, potassium, magnesium, ammonium, calcium, or ferric, and the like. Examples of suitable amines include isopropylamine, trimethylamine, histidine, N,N′ dibenzylethylenediamine, chloroprocaine, choline, diethanolamine, dicyclohexylamine, ethylenediamine, N methylglucamine, and procaine.

Pharmaceutically acceptable acid addition salts include inorganic or organic acid salts. Examples of suitable acid salts include the hydrochlorides, acetates, citrates, salicylates, nitrates, phosphates. Other suitable pharmaceutically acceptable salts are well known to those skilled in the art and include, for example, acetic, citric, oxalic, tartaric, or mandelic acids, hydrochloric acid, hydrobromic acid, sulfuric acid or phosphoric acid; with organic carboxylic, sulfonic, sulfo or phospho acids or N substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4 aminosalicylic acid, 2 phenoxybenzoic acid, 2 acetoxybenzoic acid, embonic acid, nicotinic acid or isonicotinic acid; and with amino acids, such as the 20 alpha amino acids involved in the synthesis of proteins in nature, for example glutamic acid or aspartic acid, and also with phenylacetic acid, methanesulfonic acid, ethanesulfonic acid, 2 hydroxyethanesulfonic acid, ethane 1,2 disulfonic acid, benzenesulfonic acid, 4 methylbenzenesulfoc acid, naphthalene 2 sulfonic acid, naphthalene 1,5 disulfonic acid, 2 or 3 phosphoglycerate, glucose 6 phosphate, N cyclohexylsulfamic acid (with the formation of cyclamates), or with other acid organic compounds, such as ascorbic acid.

Specifically, BH4 salts with inorganic or organic acids are preferred. Nonlimiting examples of alternative BH4 salts forms includes BH4 salts of acetic acid, citric acid, oxalic acid, tartaric acid, fumaric acid, and mandelic acid.

The frequency of BH4 dosing will depend on the pharmacokinetic parameters of the agent and the routes of administration. The optimal pharmaceutical formulation will be determined by one of skill in the art depending on the route of administration and the desired dosage. See for example Remington's Pharmaceutical Sciences, 18th Ed. (1990, Mack Publ. Co, Easton Pa. 18042) pp 1435 1712, incorporated herein by reference; Such formulations may influence the physical state, stability, rate of in vivó release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Appropriate dosages may be ascertained through the use of established assays for determining blood levels of Phe in conjunction with relevant dose response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey ducks and geese.

In certain aspects of the present invention, all the necessary components for the treatment of PPHN using BH4 either alone or in combination with another agent or intervention traditionally used for the treatment of pulmonary disease may be packaged into a kit. Specifically, the present invention provides a kit for use in the therapeutic intervention of PPHN comprising a packaged set of medicaments that comprise BH4 or a derivative or precursor thereof as well as buffers and other components for preparing deliverable forms of said medicaments, and/or devices for delivering such medicaments, and/or any agents that are used in combination therapy with such BH4-based medicaments, and/or instructions for the treatment of PPHN packaged with the medicaments. The instructions may be fixed in any tangible medium, such as printed paper, or a computer-readable magnetic or optical medium, or instructions to reference a remote computer data source such as a world wide web page accessible via the internet.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Clinical Evaluation With 6R-Tetrahydrobiopterin

The following example provides guidance on the parameters to be used for the clinical evaluation BH4 in the therapeutic methods of the present invention. As discussed herein throughout, BH4 will be used in the treatment of PPHN including primary and secondary PPHN. Clinical trials will be conducted which will provide an assessment of daily oral doses of BH4 for safety, pharmacokinetics, and initial response of both surrogate and defined clinical endpoints. The trial will be conducted for a minimum, but not necessarily limited to 1 week for each patient to assess efficacy in reversing PPHN, and to collect sufficient safety information for 30 evaluable patients.

The initial dose for the trials will vary from about 2 to about 10 mg/kg. In the event that this dose does not produce a reduction in pulmonary pressures in a patient, or produce a significant direct clinical benefit measured as an [describe], the dose should be increased as necessary, and maintained for an additional minimal period of, but necessarily limited to, 1 week to establish safety and to evaluate further efficacy. Lower doses, e.g., doses of between 0.1 to 2 mg/kg also are contemplated.

Measurements of safety will include adverse events, allergic reactions, complete clinical chemistry panel (kidney and liver function), urinalysis, and CBC with differential. In addition, other parameters including the reduction in pulmonary pressures, [list other indicia specific for. PPHN) also will be monitored. The present example also contemplates the determination of pharmacokinetic parameters of the drug in the circulation, and general distribution and half-life of 6R-BH4 in blood. It is anticipated that these measures will help relate dose to clinical response.

### Methods

Patients who have reduced arterial oxygen pressures, evidence of shunting through the patent ductus arteriosus including decreased oxygenation in the lower extremities and other symptoms of PPHN will undergo a baseline a medical history and physical exam, and various diagnostic tests commonly used to diagnose PPHN in the clinical setting including but not limited to hyperoxia, hyperoxia-hyperventiliation and echocardiography studies. The proposed human dose of 2 to about 10 mg/kg BH4 will be administered divided in one to three daily doses. Blood gases including arterial oxygen pressures will be monitored at frequent intervals of approximately every hour to every 4 hours and pulse oximetry of the right upper and left lower extremity will be monitored continuously. A complete evaluation will be conducted one week after completing the treatment period. Should dose escalation be required, the patients will follow the same schedule outlined above. Safety will be monitored throughout the trial.

Enrolled patients will be randomized to receive BH4 or a placebo first followed by the reverse placebo or active BH4 in a second administration 1 hour later. The patient with active PPHN and demonstrable shunting will be administered the drug (active or placebo) via an NGT and the child monitored for one hour by pulse oximetry and blood gases. After the 1 hour period, the patient will receive a second drug (either placebo or drug, opposite to what was received first). The patient again will be monitored. If the patient receives BH4 first, if effective, a prolonged improvement in pulmonary blood flow would be expected, as evidenced by improving oxygenation in the lower extremity and in the blood gases. An echo will be used to document the degree of patent ductus reverse flow (right to left). If after this dose, the patient receives a placebo, no change will be observed. If the patient received the placebo first, no change is expected but in the second hour when the patient then receives the active BH4, the effect should occur within the hour. By showing that this effect only occurs in infants receiving active for the first time, we can show that the patient respond to BH4 specifically. This can be done then without undue risk to sick patients. After the 2 hour period, and assuming there are no contraindications, the babies will receive BH4 divided BID for a 1 week period and continued thereafter if indicated.

### Diagnosis and Inclusion/Exclusion Criteria

The patient may be male or female, aged 0 to one month with a documented diagnosis of PPHN confirmed by echocardiography and evidence of reduced arterial oxygen pressures to less than PaO₂ 45 mmHg or an oxygen saturation of less than 94% on room air.

### Dose, Route and Regimen

Patients will receive BH4 at a dose of 5 mg/kg per day. In the event that pulmonary pressure are not decreased by a reasonable amount and no clinical benefit is observed, the dose may be increased as necessary until a total daily dose of 20 mg/kg is administered. The daily BH4 dosage will be administered orally or via nasogastric tube as liquid, powder, tablets or capsules. The total daily dose may be given as a single dose or perhaps divided in two or three daily doses. The patients will be monitored clinically as well as for any adverse reactions. If any unusual symptoms are observed, study drug administration will be stopped immediately, and a decision will be made about study continuation.

### BH4 Safety

BH4 therapy will be determined to be safe if no significant acute or chronic drug reactions occur during the course of the study. The longer-term administration of the drug will be determined to be safe if no significant abnormalities are observed in the clinical examinations, clinical labs, or other appropriate studies.

### Example 2

### Preparation of Stabilized Crystallized form of BH4

U.S. patent application Ser. No. ______, entitled “Polymorphs of (6R)-L-erythro-tetrahydrobiopterin dihydrochloride” filed on Nov. 17, 2004 in the name of Applicants Rudolf MOSER, of Schaffhausen, Switzerland and Viola GROEHN of Dachsen, Switzerland and assigned Merck-Eprova internal reference number Z7053CH00 (referred to herein as the “Moser Application” is incorporated herein by reference in its entirety as teaching methods of preparing modified BH4 compositions, characterization of the modifications, and stability data of the modified BH4 compositions. The examples of that specification describe X ray and Raman spectra studies to characterize the polymorphs of BH4. Each of the BH4 compositions of that application may be used in the treatment methods described herein. The following description provides additional background and a brief characterization of some of those exemplary compositions.

Results obtained during development of (6R)-L-erythro-tetrahydrobiopterin dihydrochloride (see “Moser Application”) indicated that the compound may possess polymorphic forms. The continued interest in this area requires an efficient and reliable method for the preparation of individual polymorphs of (6R)-L-erythro-tetrahydrobiopterin dihydrochloride and controlled crystallization conditions to provide polymorphs, which are preferably stable and easily to handle and to process in the manufacture and preparation of formulations.

Crystallization techniques well known in the art for producing drug crystals are used to prepare the prepare the polymorph forms. Such techniques include, but are not limited to, techniques such as suspension, predipitation, re-crystallization, evaporation, solvent like water sorption methods or decomposition of solvates. Diluted, saturated or super-saturated solutions of the BH4 may be used for crystallization, with or without seeding with suitable nucleating agents. Temperatures up to 150° C. may be applied to form solutions of the drug. Cooling to initiate crystallization and precipitation down to −100° C. and preferably down to −30° C. may be applied. Metastable polymorph or pseudo-polymorph forms can be used to prepare solutions or suspensions for the preparation of more stable forms and to achieve higher concentrations in the solutions.

As discussed in the Moser Application, the polymorph form may be obtained by crystallization of the BH4 from polar solvent mixtures. The Moser Application also describes a process for the preparation of polymorph form of (6R)-L-erythro-tetrahydrobiopterin dihydrochloride, comprising dissolution, optionally at elevated temperatures, of a solid lower energy form than the claimed form of (6R)-L-erythro-tetrahydrobiopterin dihydrochloride in a polar solvent mixture, addition of seeds to the solution, cooling the obtained suspension and isolation of the formed crystals.

Dissolution may be carried out at room temperature or up to 70° C., More preferably the dissolution is carried out at temperatures up to 50° C. The starting material may be added to the final solvent mixture for dissolution, or alternatively the starting material first may be dissolved in water and other solvents may than be added both or one after the other solvent. The solution of the BH4 is preferably stirred. Cooling may mean temperatures down to −80° C., preferably down to −40° C. to 0° C. In some embodiments, in order to initiate the crystallization of the BH4 polymorph, the solution may be seeded. Suitable seeds may include a portion of the polymorph form from another batch of crystals, or crystals having a similar or identical morphology. After isolation, the crystalline form can be washed with acetone or tetrahydrofurane and dried using techniques commonly used for drying drug crystals.

The polymorph forms of BH4 described in the Moser Application are a very stable crystalline form of the drug. The polymorph form can be easily filtered off, dried and ground to particle sizes desired for pharmaceutical formulations. These outstanding properties renders this polymorph form especially feasible for pharmaceutical application. The stability of the polymorph form of BH4 was determined after the BH4×2HCl (the polymorph form) had been stored for 8 months in a minigrip bag at 40° C. and 75% relative humidity. Quality was checked in different intervals throughout the 8 month period by HPLC. After 8 months, the quality and stability of the polymorph was surprisingly similar to the stability seen at time zero:

| | 0 months | | | | |
|---|---|---|---|---|---|
| | (at the | after | after | after | after |
| | beginning) | 1 week | 1 month | 3 months | 8 months |
| HPLC | 98.4 | 99.4 | 98.3 | 99.1 | 98.1 |
| [% area] | | | | | |

Accordingly, the Moser Application provides descriptions of a pharmaceutical compositions comprising a polymorph form of (6R)-L-erythro-tetrahydrobiopterin dihydrochloride and a pharmaceutically acceptable carrier or diluent. Such compositions will be useful in the therapeutic methods described herein.

In addition to the Moser Application, those of skill in the art also are referred to U.S. Pat. Nos. 6,596,721; 6,441,168; and 6,271,374 which describe various methods and compositions for producing stable crystalline salts of 5-methyltetrahydrofolic acid and methods and compositions for producing stable forms of 6R tetrandrofolic acid and methods and compositions for producing stable forms of 6S and 6R tetrandrofolic acid. Each of these patents are incorporated herein by reference in their entirety as generally teaching methods of producing crystalline forms of agents and techniques for characterizing such agents. Such methods may be used in producing stable forms of BH4 for use as pharmaceutical compositions in the treatment methods taught herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 3

### Stable Tablet Formulation of Tetrahydrobiopterin

A tablet formulation was prepared by mixing the ingredients shown in Table I as described in detail below.

TABLE I

| Ingredient | Weight Percent |
|---|---|
| 6R-L-erythro-5, 6, 7, 8-tetrahydrobiopterin | 33.33 |
| dihydrochloride salt | |
| (Active Ingredient) | |
| D-Mannitol | 57.56 |
| (Taste Masking) | |
| Dibasic Calcium Phosphate, Anhydrous | 2.18 |
| (Taste Masking) | |
| Crosprovidone | 4.50 |
| (Disintegrant) | |
| Ascorbic acid | 1.67 |
| (Stabilizer) | |
| Riboflavin | 0.01 |
| (Coloring Agent) | |
| Sodium Stearyl Fumarate | 0.75 |
| (Lubricant) | |

A twelve kilogram batch of a pharmaceutical preparation of BH4 and the excipients listed in Table I was prepared by first charging 4 kg of 6R-L-erythro-5, 6, 7, 8-tetrahydrobiopterin dihydrochloride salt (Sapropterin Hydrochloride, available from Daiichi Suntory Pharma Co., Ltd., Japan to a blender and blending the BH4 for 10 minutes at 25 revolutions per minute (RPM). Then 6.91 kg of D-Mannitol (PEARLITOL, available from Roquette America, Inc., Keokuk, Iowa) was added to the blender and the mixture was allowed to blend for an additional 10 minutes at 25 RPM. Then 260 grams of Anhydrous Dibasic Calcium Phosphate (available from Mallinckrodt Baker, Inc., Phillipsburg, N.J.) and 540 grams of Polyvinylpyrrolidone (KOLLIDON CL, available from BASF Corporation, Florham Park, N.J.) were added to the blender and the mixture was allowed to blend for an additional 10 minutes at 25 RPM. To the bender 200 grams of Ascorbic Acid and 120 grams of Ribofloavin were added to the blender and the mixture was allowed to blend for 3 minutes at 25 RPM. The Sodium Stearyl Fumarate lubricant (PRUV, available from Penwest Pharmaceuticals Co., Danbury, Conn.) was filtered through a 25 mesh stainless steel screen and into a bag, and the blender was then charged with 9 kg of the screened Sodium Stearyl Fumarate, and the resulting mixture was allowed to blend for 5 minutes at 25 RPM.

The blended mixture was then removed from the blender, and three samples were collected for the preparation of a 150 mg, a 300 mg, and a 600 mg tablets. The 12 kg batch material prepared as described above was placed in a tablet press (available from Jenn-Chiang Mahinery Co., Ltd., Taiwan, R.O.C.) wherein the parameters of the tablet press were set to provide tablets with a thickness in the range of 4.5 to 5.5 millimeters, and a target hardness of 7 KP.

The resulting tablets were then analyzed to determine the stability of the formulation. The stability of the formulation was studied for a change in appearance over time by a visual inspection at different intervals, for disintegration of the formulation utilizing the United States Pharmacopeia recommendations no. 701, and for a chemical change by assaying the components of the formulation. The results of the stability tests are summarized below in Table II.

TABLE II

| Test | Initial | 2 weeks | 4 weeks | 8 weeks |
|---|---|---|---|---|
| Appear- | Color is | Rough surface, | Rough surface, | Rough surface, |
| ance | off white | and color is | and color is | and color is |
| | | light yellow | light yellow | yellow |
| Disinte- | 1 min | 35 sec | 58 sec | — |
| gration | 52 sec | | | |
| Chemical | 100.20% | 102.90% | 97.4 | 99.8 |
| Assay | | | | |

These stability test confirm that the resulting tablet formulation is stable and useful in a pharmaceutical preparation of BH4 disclosed herein. Other suitable tablet formulations may include at least ascorbic acid at a concentration of at least 0.01% weight, or at least 0.05% weight or at least 0.1% weight, and optionally a disintegrant (preferably crossprovidone).

What is claimed is:

1. A method for treating an infant having below normal arterial oxygen pressure (PaO₂) comprising administering to said subject a composition comprising tetrahydrobiopterin (BH4) or a precursor or derivative thereof, wherein the administration of BH4 is administered in an amount effective to increase PaO₂ of said infant as compared to said PaO₂ in the absence of said administration of BH4.

2. The method of claim 1, wherein said infant is between the ages of less than 34 weeks gestational age and about one month post-natal agent.

3. The method of claim 1, wherein said infant has been diagnosed as having a Persistent Pulmonary Hypertension of the Newborn (PPHN).

4. The method of claim 1, wherein said infant has been diagnosed with primary PPHN, secondary PPHN, PPHN associated with hypoplastic lungs.

5. A method for treating Persistent Pulmonary Hypertension of the Newborn (PPHN) in a subject comprising administering to said subject a composition comprising tetrahydrobiopterin (BH4) or a precursor or derivative thereof, wherein the administration of BH4 is effective to increasing arterial oxygen pressure of said subject as compared to said arterial oxygen pressure in the absence of said BH4 administration.

6. The method of any of claims 1 to 5, wherein said subject has an arterial oxygen pressure (PaO₂) of less than 45 mm Hg in the absence of a therapeutic regimen.

7. The method of any of claims 1 to 5, wherein said subject has a PaO₂ of less than 45 mmHg and greater than 15 mmHg difference between preductal PaO₂ and postductal PaO₂ when placed on 100% O₂ in the absence of a therapeutic regimen.

8. The method of any of claims 1 to 5, wherein said subject has a PaO₂ of 100 mmHg when hyperinflated with a manual resuscitator and placed on 100% O₂ until arterial carbon dioxide (PaCO₂) is between 20 to 25 mm Hg in the absence of a therapeutic regimen.

9. The method of any of claims 1 to 5, wherein said subject has a PaO₂ of less than 100 mmHg when hyperinflated with a manual resuscitator and placed on 100% O₂ until arterial carbon dioxide (PaCO₂) is between 20 to 25 mm Hg and a normal echo lacking evidence of congenital heart disease wherein said subject is assessed by echocardiography in the absence of a therapeutic regimen.

10. The method of any of claims 1 to 5, wherein said subject has a right ventricular pre-ejection period (PEP) to ejection time (ET) ratio of greater than 0.50 and left ventricular PEP/ET ratio of greater than 0.38 when said subject is assessed by echocardiography in the absence of a therapeutic regimen.

11. The method of any of claims 5 to 10, wherein said BH4 administration increases PaO₂ of said subject to greater than 45 mm Hg.

12. The method of any of claims 5 to 10, wherein said BH4 administration increases PaO₂ of said subject to between about 45 mm Hg to about 120 mmHg.

13. The method of any of claims 5 to 10, wherein said BH4 administration increases PaO₂ of said subject to between about 45 mm Hg to about 65 mmHg, wherein said subject is a preterm infant with PPHN and is less than 37 weeks gestational age.

14. The method of any of claims 5 to 10, wherein said BH4 administration increases PaO₂ of said subject to between about 50 mm Hg to about 70 mmHg, wherein said subject is a full term infant between 37 and 42 weeks gestational age or is a post term infant at 42 weeks or greater) with PPHN of between 0 and one month of post-natal age.

15. The method of any of claims 5 to 10, wherein said BH4 administration increases PaO₂ of said subject to between about 50 mm Hg to about 70 mmHg, wherein said subject is a post term infant born two weeks or more after 280 days of gestation.

16. The method of claim 1, wherein said BH4 is administered in an amount of between about 0.1 mg/kg to about 30 mg/kg.

17. The method of claim 16, wherein said BH4 is administered in a single daily dose.

18. The method of claim 16, wherein said BH4 is administered in multiple doses on a daily basis.

19. The method of claim 16, wherein said BH4 is administered on a daily basis until PaO₂ of said subject is increased to greater than 45 mm Hg.

20. The method of claim 16, wherein said BH4 is administered on a daily basis until PaO₂ of said subject is increased to between about 45 mm Hg and about 120 mmHg.

21. The method of claim 16, wherein said BH4 is administered on a daily basis until PaO₂ of said subject is increased to between about 45 mm Hg to about 65 mmHg, wherein said subject is a preterm infant with PPHN and is less than 37 weeks gestational age.

22. The method of claim 16, wherein said BH4 is administered on a daily basis until PaO₂ of said subject is increased to between about 50 mm Hg to about 70 mmHg, wherein said subject is a full term infant with PPHN and is between 37 and about 41 weeks gestational age.

23. The method of claim 16, wherein said BH4 is administered on a daily basis until PaO₂ of said subject is increased to between about 50 mm Hg to about 70 mmHg, wherein said subject is a post term infant with PPHN and is born two weeks or more after 280 days of gestation.

24. The method of claim 16, wherein the PaO₂ of said subject is monitored on a daily basis and said BH4 is administered when a 10 mm Hg or 20% increase in PaO₂ is observed.

25. The method of claim 1, wherein said BH4 is administered as a stabilized crystallized form.

26. The method of claim 25, wherein said stabilized crystallized form of BH4 comprises at least 99.5% pure 6R BH4.

27. The method of claim 25, wherein said stabilized BH4 composition is stable at room temperature for more than 8 hours.

28. The method of claim 1, wherein said BH4 precursor is dihydrobiopterin (BH2).

29. The method of claim 1, wherein said BH4 precursor is sepiapterin.

30. The method of claim 1, wherein said BH4 is administered orally.

31. The method of claim 1, wherein BH4 is administered in combination with an agent or intervention used to treat PPHN.

32. The method of claim 31, wherein said agent is a vasodilator.

33. The method of claim 32, wherein said vasodilator is selected from the group consisting of tolazoline, magnesium sulphate, nitroprusside, prostacyclin, dipyramidole, adenosine triphosphate, and inhaled nitric oxide.

34. The method of any of claims 1 to 33, wherein said BH4 comprises a crystal form of BH4 selected from the group consisting of crystal polymorph form A, crystal polymorph form B, crystal polymorph form F, crystal polymorph form J, crystal polymorph form K, crystal hydrate form C, crystal hydrate form D, crystal hydrate form E, crystal hydrate form H, crystal hydrate form O, solvate crystal form G, solvate crystal form I, solvate crystal form L, solvate crystal form M, solvate crystal form N, and combinations thereof.

35. The method of claim 34, wherein said composition further comprises a folate.

36. The method of claim 35, wherein said folate comprises a tetrahydrofolate selected from the group consisting of tetrahydrofolate is 5-formyl-(6S)-tetrahydrofolic acid and salts thereof, 5-methyl-(6S)-tetrahydrofolic acid and salts thereof, 5,10-methylene-(6R)-tetrahydrofolic acid and salts thereof, 5,10-methenyl-(6R)-tetrahydrofolic acid and salts thereof, 10-formyl-(6R)-tetrahydrofolic acid, 5-formimino-(6S)-tetrahydrofolic acid salts thereof, (6S)-tetrahydrofolic acid and salts thereof, and combinations of the foregoing.

37. The method of claim 35, wherein said composition further comprises arginine.

38. Use of a composition comprising BH4, or a precursor or derivative thereof for the manufacture of a medicament for the treatment of below normal arterial oxygen pressure (PaO₂) in an infant.

39. The use of claim 38, wherein said infant is between the ages of less than 34 weeks gestational age and about one month post-natal age.

40. The use of claim 38, wherein said medicament is for the treatment of infant that has been diagnosed as having PPHN.

41. The use of claim 38, wherein said medicament is for the treatment of infant that has been diagnosed as having primary PPHN, secondary PPHN, or PPHN associated with hypoplastic lungs.

42. Use of a composition comprising BH4, or a precursor or derivative thereof for the manufacture of a medicament for the treatment PPHN.

43. Use of a composition comprising BH4, or a precursor or derivative thereof for the manufacture of a medicament for increasing arterial oxygen pressure of a subject as compared to said arterial oxygen pressure in the absence of said BH4 administration.

44. A use of any of claims 38 through 43, wherein said medicament is formulated for administration as a single daily dose.

45. A use of any of claims 38 through 43, wherein said medicament is formulated for administration as multiple daily doses.

46. A use of any of claims 38 through 43, wherein said medicament is formulated as an inhalable formulation.

47. A use of any of claim 38 through 43 wherein said medicament is formulated to deliver a dose of from about 0.1 mg/kg to about 30 mg/kg per day.

48. A use of any of claims 38 through 43, wherein said medicament is prepared using a stabilized crystallized form of BH4.

49. The use of claim 48, wherein said stabilized crystallized form of BH4 comprises at least 99.5% pure 6R BH4.

50. The use of claims 48, wherein said stabilized BH4 composition is stable at room temperature for more than 8 hours.

51. The use of any of claims 38 through 47, wherein said BH4 precursor in said medicament is dihydrobiopterin (BH2).

52. The use of any of claims 38 through 47, wherein said BH4 precursor in said medicament is sepiapterin.

53. The use of any of claims 38 through 47, wherein said medicament is provided for use in combination therapy with an agent or intervention used in the treatment of PPHN.

54. The use of claim 53, wherein said agent is a vasodilator.

55. The use of claim 53, wherein said vasodilator is selected from the group consisting of tolazoline, magnesium sulphate, nitroprusside, prostacyclin, dipyramidole, adenosine triphosphate, and inhaled nitric oxide.

56. The use of any of claims 38 through 55, wherein said BH4 comprises a crystal form of BH4 selected from the group consisting of crystal polymorph form A, crystal polymorph form B, crystal polymorph form F, crystal polymorph form J, crystal polymorph form K, crystal hydrate form C, crystal hydrate form D, crystal hydrate form E, crystal hydrate form H, crystal hydrate form O, solvate crystal form G, solvate crystal form I, solvate crystal form L, solvate crystal form M, solvate crystal form N, and combinations thereof.

57. The use of claim 56, wherein said medicament further comprises a folate.

58. The use of claim 57, wherein said folate comprises a tetrahydrofolate selected from the group consisting of tetrahydrofolate is 5-formyl-(6S)-tetrahydrofolic acid and salts thereof, 5-methyl-(6S)-tetrahydrofolic acid and salts thereof, 5,10-methylene-(6R)-tetrahydrofolic acid and salts thereof, 5,10-methenyl-(6R)-tetrahydrofolic acid and salts thereof, 10-formyl-(6R)-tetrahydrofolic acid, 5-formimino-(6S)-tetrahydrofolic acid salts thereof, (6S)-tetrahydrofolic acid and salts thereof, and combinations of the foregoing.

59. The use of claim 56, wherein said composition further comprises arginine.

60. A kit comprising a medicament of any of claims 37-59, and instructions for the treatment of PPHN and optionally a device for the delivery of said medicament.

61. A composition comprising BH4, or a precursor or derivative thereof for the manufacture of a medicament for the treatment of below normal arterial oxygen pressure (PaO₂) in an infant.

62. The composition of claim 61, wherein said infant is between the ages of less than 34 weeks gestational age and about one month post-natal age.

63. The composition of claim 61, wherein said medicament is for the treatment of infant that has been diagnosed as having PPHN.

64. The composition of claim 61, wherein said medicament is for the treatment of infant that has been diagnosed as having primary PPHN, secondary PPHN, or PPHN associated with hypoplastic lungs.

65. A composition comprising BH4, or a precursor or derivative thereof for the manufacture of a medicament for the treatment PPHN.

66. A composition comprising BH4, or a precursor or derivative thereof for the manufacture of a medicament for increasing arterial oxygen pressure of a subject as compared to said arterial oxygen pressure in the absence of said BH4 administration.

67. The composition of any of claims 61 through 66, wherein said medicament is formulated for administration as a single daily dose.

68. The composition of any of claims 61 through 66, wherein said medicament is formulated for administration as multiple daily doses.

69. The composition of any of claims 61 through 66, wherein said medicament is formulated as an inhalable formulation.

70. The composition of any of claims 61 through 66, wherein said medicament is formulated to deliver a dose of from about 0.1 mg/kg to about 30 mg/kg per day.

71. The composition of any of claims 61 through 66, wherein said medicament is prepared using a stabilized crystallized form of BH4.

72. The composition of claim 71, wherein said stabilized crystallized form of BH4 comprises at least 99.5% pure 6R BH4.

73. The composition of claim 71, wherein said stabilized BH4 composition is stable at room temperature for more than 8 hours.

74. The composition of any of claims 61 through 73, wherein said BH4 precursor in said medicament is dihydrobiopterin (BH2).

75. The composition of any of claims 61 through 73, wherein said BH4 precursor in said medicament is sepiapterin.

76. The composition of any of claims 61 through 73, wherein said medicament is provided for use in combination therapy with an agent or intervention used in the treatment of PPHN.

77. The composition of claim 76, wherein said agent is a vasodilator.

78. The composition of claim 77, wherein said vasodilator is selected from the group consisting of tolazoline, magnesium sulphate, nitroprusside, prostacyclin, dipyramidole, adenosine triphosphate, and inhaled nitric oxide.

79. The composition of any of claims 61 through 78, wherein said BH4 comprises a crystal form of BH4 selected from the group consisting of crystal polymorph form A, crystal polymorph form B, crystal polymorph form F, crystal polymorph form J, crystal polymorph form K, crystal hydrate form C, crystal hydrate form D, crystal hydrate form E, crystal hydrate form H, crystal hydrate form O, solvate crystal form G, solvate crystal form I, solvate crystal form L, solvate crystal form M, solvate crystal form N, and combinations thereof.

80. The composition of claim 79, wherein said medicament further comprises a folate.

81. The composition of claim 80, wherein said folate comprises a tetrahydrofolate selected from the group consisting of tetrahydrofolate is 5-formyl-(6S)-tetrahydrofolic acid and salts thereof, 5-methyl-(6S)-tetrahydrofolic acid and salts thereof, 5,10-methylene-(6R)-tetrahydrofolic acid and salts thereof, 5,10-methenyl-(6R)-tetrahydrofolic acid and salts thereof, 10-formyl-(6R)-tetrahydrofolic acid, 5-formimino-(6S)-tetrahydrofolic acid salts thereof, (6S)-tetrahydrofolic acid and salts thereof, and combinations of the foregoing.

82. The composition of claim 61, wherein said composition further comprises arginine.
